SEQUENCE LISTING (352 sequences)

SEQ ID NO: 1 - protein, 8 aa, Artificial Sequence
  LOS peptide mimotope sequence
NTKWYPYA

SEQ ID NO: 2 - protein, 9 aa, Artificial Sequence
  LOS peptide mimotope sequence
FVPSPYVYE

SEQ ID NO: 3 - protein, 7 aa, Artificial Sequence
  LOS peptide mimotope sequence
RSSLPGD

SEQ ID NO: 4 - protein, 9 aa, Artificial Sequence
  LOS peptide mimotope sequence
FYRELAGDL

SEQ ID NO: 5 - protein, 9 aa, Artificial Sequence
  LOS peptide mimotope sequence
MRRTASEIM

SEQ ID NO: 6 - protein, 9 aa, Artificial Sequence
  LOS peptide mimotope sequence
MRPLTWQTT

SEQ ID NO: 7 - protein, 9 aa, Artificial Sequence
  LOS peptide mimotope sequence
RMRIIPEGT

SEQ ID NO: 8 - protein, 9 aa, Artificial Sequence
  LOS peptide mimotope sequence
MRDVMPQHW

SEQ ID NO: 9 - protein, 9 aa, Artificial Sequence
  LOS peptide mimotope sequence
HKPTDHPSW

SEQ ID NO: 10 - protein, 9 aa, Artificial Sequence
  LOS peptide mimotope sequence
SETYGRPGL

SEQ ID NO: 11 - protein, 9 aa, Artificial Sequence
  LOS peptide mimotope sequence
ERPIGGDSG

SEQ ID NO: 12 - protein, 9 aa, Artificial Sequence
  LOS peptide mimotope sequence
RMRDIPGAP

SEQ ID NO: 13 - protein, 9 aa, Artificial Sequence
  LOS peptide mimotope sequence
ISEYAKGTT

SEQ ID NO: 14 - protein, 9 aa, Artificial Sequence
  LOS peptide mimotope sequence
SHAPPYDRV

SEQ ID NO: 15 - protein, 9 aa, Artificial Sequence
  LOS peptide mimotope sequence
VTIPYRGTQ

SEQ ID NO: 16 - protein, 9 aa, Artificial Sequence
  LOS peptide mimotope sequence
FAPPYDPLP

SEQ ID NO: 17 - protein, 9 aa, Artificial Sequence
  LOS peptide mimotope sequence
APYSIFIGE

SEQ ID NO: 18 - protein, 9 aa, Artificial Sequence
  LOS peptide mimotope sequence
THLYHYGTS

SEQ ID NO: 19 - protein, 9 aa, Artificial Sequence
  LOS peptide mimotope sequence
LCQAYKGRR

SEQ ID NO: 20 - protein, 7 aa, Artificial Sequence
  LOS peptide mimotope sequence
DPRLLDL

SEQ ID NO: 21 - protein, 9 aa, Artificial Sequence
  LOS peptide mimotope sequence
KTALPPYDR

SEQ ID NO: 22 - protein, 9 aa, Artificial Sequence
  LOS peptide mimotope sequence
FARPFQGTW

SEQ ID NO: 23 - protein, 9 aa, Artificial Sequence
  LOS peptide mimotope sequence
SLSLPPYDR

SEQ ID NO: 24 - protein, 9 aa, Artificial Sequence
  LOS peptide mimotope sequence
DRTLSALAL

SEQ ID NO: 25 - protein, 9 aa, Artificial Sequence
  LOS peptide mimotope sequence
RAPPYDTIM

SEQ ID NO: 26 - protein, 11 aa, Artificial Sequence
  LOS peptide mimotope sequence
CPPYDEGCRVA

SEQ ID NO: 27 - protein, 9 aa, Artificial Sequence
  LOS peptide mimotope sequence
FGLIAFHPD

SEQ ID NO: 28 - protein, 9 aa, Artificial Sequence
  LOS peptide mimotope sequence
QPIGPPYDR

SEQ ID NO: 29 - protein, 9 aa, Artificial Sequence
  LOS peptide mimotope sequence
TANYYFGTY

SEQ ID NO: 30 - protein, 9 aa, Artificial Sequence
  LOS peptide mimotope sequence
ANSRPGGYL

SEQ ID NO: 31 - protein, 9 aa, Artificial Sequence
  LOS peptide mimotope sequence
MSSYGRGVR

SEQ ID NO: 32 - protein, 9 aa, Artificial Sequence
  LOS peptide mimotope sequence
VSTPFRGTF

SEQ ID NO: 33 - protein, 9 aa, Artificial Sequence
  LOS peptide mimotope sequence
DPRITPDFG

SEQ ID NO: 34 - protein, 9 aa, Artificial Sequence
  LOS peptide mimotope sequence
GPPYDPFPA

SEQ ID NO: 35 - protein, 9 aa, Artificial Sequence
  LOS peptide mimotope sequence
HTVRFRGTL

SEQ ID NO: 36 - protein, 9 aa, Artificial Sequence
  LOS peptide mimotope sequence
TAPPYDAYG

SEQ ID NO: 37 - protein, 9 aa, Artificial Sequence
  LOS peptide mimotope sequence
RSPLLGAPV

SEQ ID NO: 38 - protein, 9 aa, Artificial Sequence
  LOS peptide mimotope sequence
TTTYGTGTW

SEQ ID NO: 39 - protein, 9 aa, Artificial Sequence
  LOS peptide mimotope sequence
SSLYYHGTA

SEQ ID NO: 40 - protein, 9 aa, Artificial Sequence
  LOS peptide mimotope sequence
LYEPLRGTL

SEQ ID NO: 41 - protein, 9 aa, Artificial Sequence
  LOS peptide mimotope sequence
APPPYDQSF

SEQ ID NO: 42 - protein, 9 aa, Artificial Sequence
  LOS peptide mimotope sequence
PPPWYSRSS

SEQ ID NO: 43 - protein, 9 aa, Artificial Sequence
  LOS peptide mimotope sequence
SRALGYVSE

SEQ ID NO: 44 - protein, 9 aa, Artificial Sequence
  LOS peptide mimotope sequence
PTWYKLKSV

SEQ ID NO: 45 - protein, 9 aa, Artificial Sequence
  LOS peptide mimotope sequence
RGSEGSFAR

SEQ ID NO: 46 - protein, 9 aa, Artificial Sequence
  LOS peptide mimotope sequence
KQTIGSFDG

SEQ ID NO: 47 - protein, 9 aa, Artificial Sequence
  LOS peptide mimotope sequence
PLWYDPAPP

SEQ ID NO: 48 - protein, 9 aa, Artificial Sequence
  LOS peptide mimotope sequence
ESPYSAHRW

SEQ ID NO: 49 - protein, 9 aa, Artificial Sequence
  LOS peptide mimotope sequence
WYDERTILK

SEQ ID NO: 50 - protein, 9 aa, Artificial Sequence
  LOS peptide mimotope sequence
SSYSYVHDS

SEQ ID NO: 51 - protein, 9 aa, Artificial Sequence
  LOS peptide mimotope sequence
RFTYDPPFM

SEQ ID NO: 52 - protein, 9 aa, Artificial Sequence
  LOS peptide mimotope sequence
RLYSFVFDK

SEQ ID NO: 53 - protein, 9 aa, Artificial Sequence
  LOS peptide mimotope sequence
SQWRSAAPT

SEQ ID NO: 54 - protein, 9 aa, Artificial Sequence
  LOS peptide mimotope sequence
RPAFDPPYH

SEQ ID NO: 55 - protein, 9 aa, Artificial Sequence
  LOS peptide mimotope sequence
HGRTLWYTP

SEQ ID NO: 56 - protein, 9 aa, Artificial Sequence
  LOS peptide mimotope sequence
SSVSATYPI

SEQ ID NO: 57 - protein, 9 aa, Artificial Sequence
  LOS peptide mimotope sequence
SLVQSPKRF

SEQ ID NO: 58 - protein, 9 aa, Artificial Sequence
  LOS peptide mimotope sequence
SNWYENTPT

SEQ ID NO: 59 - protein, 9 aa, Artificial Sequence
  LOS peptide mimotope sequence
PRPGWGQSA

SEQ ID NO: 60 - protein, 11 aa, Artificial Sequence
  LOS peptide mimotope sequence
CTDPRGCGMFA

SEQ ID NO: 61 - protein, 9 aa, Artificial Sequence
  LOS peptide mimotope sequence
PRPHFGAPP

SEQ ID NO: 62 - protein, 9 aa, Artificial Sequence
  LOS peptide mimotope sequence
VTRATYPSW

SEQ ID NO: 63 - protein, 9 aa, Artificial Sequence
  LOS peptide mimotope sequence
WYIAPRKTL

SEQ ID NO: 64 - protein, 9 aa, Artificial Sequence
  LOS peptide mimotope sequence
YGYSALRDT

SEQ ID NO: 65 - protein, 9 aa, Artificial Sequence
  LOS peptide mimotope sequence
IITGSGWYV

SEQ ID NO: 66 - protein, 9 aa, Artificial Sequence
  LOS peptide mimotope sequence
THYSFYGDI

SEQ ID NO: 67 - protein, 9 aa, Artificial Sequence
  LOS peptide mimotope sequence
HWYSTEAAW

SEQ ID NO: 68 - protein, 9 aa, Artificial Sequence
  LOS peptide mimotope sequence
HRIAQSLPQ

SEQ ID NO: 69 - protein, 9 aa, Artificial Sequence
  LOS peptide mimotope sequence
ALYRFAADS

SEQ ID NO: 70 - protein, 9 aa, Artificial Sequence
  LOS peptide mimotope sequence
RPQFDPPND

SEQ ID NO: 71 - protein, 9 aa, Artificial Sequence
  LOS peptide mimotope sequence
HPALARWPL

SEQ ID NO: 72 - protein, 9 aa, Artificial Sequence
  LOS peptide mimotope sequence
QPKSLWYSV

SEQ ID NO: 73 - protein, 9 aa, Artificial Sequence
  LOS peptide mimotope sequence
RGYSHVSDA

SEQ ID NO: 74 - protein, 9 aa, Artificial Sequence
  LOS peptide mimotope sequence
DPVRTIYPI

SEQ ID NO: 75 - protein, 9 aa, Artificial Sequence
  LOS peptide mimotope sequence
WYTTPTRPV

SEQ ID NO: 76 - protein, 9 aa, Artificial Sequence
  LOS peptide mimotope sequence
QRQSLWYSS

SEQ ID NO: 77 - protein, 9 aa, Artificial Sequence
  LOS peptide mimotope sequence
NPDYSSPHE

SEQ ID NO: 78 - protein, 9 aa, Artificial Sequence
  LOS peptide mimotope sequence
PPWYPEHKT

SEQ ID NO: 79 - protein, 9 aa, Artificial Sequence
  LOS peptide mimotope sequence
ASLGLAKTT

SEQ ID NO: 80 - protein, 9 aa, Artificial Sequence
  LOS peptide mimotope sequence
WYVDGPLAT

SEQ ID NO: 81 - protein, 9 aa, Artificial Sequence
  LOS peptide mimotope sequence
RGWYADPSA

SEQ ID NO: 82 - protein, 9 aa, Artificial Sequence
  LOS peptide mimotope sequence
LWRPIDPFL

SEQ ID NO: 83 - protein, 9 aa, Artificial Sequence
  LOS peptide mimotope sequence
AERSLWYYP

SEQ ID NO: 84 - protein, 9 aa, Artificial Sequence
  LOS peptide mimotope sequence
PPWYNQSEL

SEQ ID NO: 85 - protein, 9 aa, Artificial Sequence
  LOS peptide mimotope sequence
DSAPAVKSS

SEQ ID NO: 86 - protein, 9 aa, Artificial Sequence
  LOS peptide mimotope sequence
PGWYDAHPT

SEQ ID NO: 87 - protein, 9 aa, Artificial Sequence
  LOS peptide mimotope sequence
RGLQGHIAY

SEQ ID NO: 88 - protein, 9 aa, Artificial Sequence
  LOS peptide mimotope sequence
WYSAPENAL

SEQ ID NO: 89 - protein, 9 aa, Artificial Sequence
  LOS peptide mimotope sequence
WYTAPSLSL

SEQ ID NO: 90 - protein, 9 aa, Artificial Sequence
  LOS peptide mimotope sequence
WYTNPSIAA

SEQ ID NO: 91 - protein, 8 aa, Artificial Sequence
  LOS peptide mimotope sequence
WYFSNELG

SEQ ID NO: 92 - protein, 9 aa, Artificial Sequence
  LOS peptide mimotope sequence
WYTLDIGPT

SEQ ID NO: 93 - protein, 9 aa, Artificial Sequence
  LOS peptide mimotope sequence
GPWHGPSSS

SEQ ID NO: 94 - protein, 9 aa, Artificial Sequence
  LOS peptide mimotope sequence
GDWPPFSAP

SEQ ID NO: 95 - protein, 9 aa, Artificial Sequence
  LOS peptide mimotope sequence
WYVGSVRSQ

SEQ ID NO: 96 - protein, 8 aa, Artificial Sequence
  LOS peptide mimotope sequence
ALDIAGGY

SEQ ID NO: 97 - protein, 9 aa, Artificial Sequence
  LOS peptide mimotope sequence
PPPSRGGYI

SEQ ID NO: 98 - protein, 9 aa, Artificial Sequence
  LOS peptide mimotope sequence
QAFDTSWTA

SEQ ID NO: 99 - protein, 9 aa, Artificial Sequence
  LOS peptide mimotope sequence
FLPCRRCGS

SEQ ID NO: 100 - protein, 9 aa, Artificial Sequence
  LOS peptide mimotope sequence
RPWQTAHFA

SEQ ID NO: 101 - protein, 9 aa, Artificial Sequence
  LOS peptide mimotope sequence
GQYSSSPFP

SEQ ID NO: 102 - protein, 9 aa, Artificial Sequence
  LOS peptide mimotope sequence
GRPGPYPAD

SEQ ID NO: 103 - protein, 9 aa, Artificial Sequence
  LOS peptide mimotope sequence
TPLPDGGIL

SEQ ID NO: 104 - protein, 9 aa, Artificial Sequence
  LOS peptide mimotope sequence
LKWGDGSSA

SEQ ID NO: 105 - protein, 9 aa, Artificial Sequence
  LOS peptide mimotope sequence
YPQLSHANP

SEQ ID NO: 106 - protein, 9 aa, Artificial Sequence
  LOS peptide mimotope sequence
SAYHRSLGA

SEQ ID NO: 107 - protein, 9 aa, Artificial Sequence
  LOS peptide mimotope sequence
FPLPSREFA

SEQ ID NO: 108 - protein, 9 aa, Artificial Sequence
  LOS peptide mimotope sequence
YRQSRSSWP

SEQ ID NO: 109 - protein, 9 aa, Artificial Sequence
  LOS peptide mimotope sequence
SHRFDALRR

SEQ ID NO: 110 - protein, 9 aa, Artificial Sequence
  LOS peptide mimotope sequence
VRFPDGSHS

SEQ ID NO: 111 - protein, 9 aa, Artificial Sequence
  LOS peptide mimotope sequence
SPAAFSDRL

SEQ ID NO: 112 - protein, 9 aa, Artificial Sequence
  LOS peptide mimotope sequence
VTDQWGGYL

SEQ ID NO: 113 - protein, 9 aa, Artificial Sequence
  LOS peptide mimotope sequence
VPSGRSPNT

SEQ ID NO: 114 - protein, 9 aa, Artificial Sequence
  LOS peptide mimotope sequence
PKWSDKRPQ

SEQ ID NO: 115 - protein, 9 aa, Artificial Sequence
  LOS peptide mimotope sequence
APPGIAVRT

SEQ ID NO: 116 - protein, 9 aa, Artificial Sequence
  LOS peptide mimotope sequence
MKWGPNSHS

SEQ ID NO: 117 - protein, 9 aa, Artificial Sequence
  LOS peptide mimotope sequence
LQDRAGGYL

SEQ ID NO: 118 - protein, 11 aa, Artificial Sequence
  LOS peptide mimotope sequence
CGRLEGRCSHA

SEQ ID NO: 119 - protein, 9 aa, Artificial Sequence
  LOS peptide mimotope sequence
YFIAKHGWA

SEQ ID NO: 120 - protein, 9 aa, Artificial Sequence
  LOS peptide mimotope sequence
PPRSSRGFL

SEQ ID NO: 121 - protein, 9 aa, Artificial Sequence
  LOS peptide mimotope sequence
TGISTGEYL

SEQ ID NO: 122 - protein, 9 aa, Artificial Sequence
  LOS peptide mimotope sequence
GPVFYATGL

SEQ ID NO: 123 - protein, 9 aa, Artificial Sequence
  LOS peptide mimotope sequence
LSQYADWTY

SEQ ID NO: 124 - protein, 9 aa, Artificial Sequence
  LOS peptide mimotope sequence
ARWYPISQT

SEQ ID NO: 125 - protein, 9 aa, Artificial Sequence
  LOS peptide mimotope sequence
QGFPGAPQD

SEQ ID NO: 126 - protein, 9 aa, Artificial Sequence
  LOS peptide mimotope sequence
WHFRTFPAT

SEQ ID NO: 127 - protein, 9 aa, Artificial Sequence
  LOS peptide mimotope sequence
TRRPFDPPA

SEQ ID NO: 128 - protein, 10 aa, Artificial Sequence
  LOS peptide mimotope sequence
CASPLGPCFW

SEQ ID NO: 129 - protein, 9 aa, Artificial Sequence
  LOS peptide mimotope sequence
WTDTYGDLL

SEQ ID NO: 130 - protein, 9 aa, Artificial Sequence
  LOS peptide mimotope sequence
ISAGPESSH

SEQ ID NO: 131 - protein, 9 aa, Artificial Sequence
  LOS peptide mimotope sequence
HSVQPATRA

SEQ ID NO: 132 - protein, 9 aa, Artificial Sequence
  LOS peptide mimotope sequence
PKAPFSPFK

SEQ ID NO: 133 - protein, 9 aa, Artificial Sequence
  LOS peptide mimotope sequence
IDAGSHGWL

SEQ ID NO: 134 - protein, 9 aa, Artificial Sequence
  LOS peptide mimotope sequence
RRGSPLSRY

SEQ ID NO: 135 - protein, 9 aa, Artificial Sequence
  LOS peptide mimotope sequence
SWDEIIDLG

SEQ ID NO: 136 - protein, 9 aa, Artificial Sequence
  LOS peptide mimotope sequence
RSPAGEWSS

SEQ ID NO: 137 - protein, 9 aa, Artificial Sequence
  LOS peptide mimotope sequence
AYHVLRYSA

SEQ ID NO: 138 - protein, 9 aa, Artificial Sequence
  LOS peptide mimotope sequence
AKTVRGDYY

SEQ ID NO: 139 - protein, 9 aa, Artificial Sequence
  LOS peptide mimotope sequence
LAASADTAA

SEQ ID NO: 140 - protein, 9 aa, Artificial Sequence
  LOS peptide mimotope sequence
PYTSWAREG

SEQ ID NO: 141 - protein, 10 aa, Artificial Sequence
  LOS peptide mimotope sequence
CNTKWYPYAC

SEQ ID NO: 142 - protein, 11 aa, Artificial Sequence
  LOS peptide mimotope sequence
CFVPSPYVYEC

SEQ ID NO: 143 - protein, 9 aa, Artificial Sequence
  LOS peptide mimotope sequence
CRSSLPGDC

SEQ ID NO: 144 - protein, 11 aa, Artificial Sequence
  LOS peptide mimotope sequence
CFYRELAGDLC

SEQ ID NO: 145 - protein, 11 aa, Artificial Sequence
  LOS peptide mimotope sequence
CMRRTASEIMC

SEQ ID NO: 146 - protein, 11 aa, Artificial Sequence
  LOS peptide mimotope sequence
CMRPLTWQTTC

SEQ ID NO: 147 - protein, 11 aa, Artificial Sequence
  LOS peptide mimotope sequence
CRMRIIPEGTC

SEQ ID NO: 148 - protein, 11 aa, Artificial Sequence
  LOS peptide mimotope sequence
CMRDVMPQHWC

SEQ ID NO: 149 - protein, 11 aa, Artificial Sequence
  LOS peptide mimotope sequence
CHKPTDHPSWC

SEQ ID NO: 150 - protein, 11 aa, Artificial Sequence
  LOS peptide mimotope sequence
CSETYGRPGLC

SEQ ID NO: 151 - protein, 11 aa, Artificial Sequence
  LOS peptide mimotope sequence
CERPIGGDSGC

SEQ ID NO: 152 - protein, 11 aa, Artificial Sequence
  LOS peptide mimotope sequence
CRMRDIPGAPC

SEQ ID NO: 153 - protein, 11 aa, Artificial Sequence
  LOS peptide mimotope sequence
CISEYAKGTTC

SEQ ID NO: 154 - protein, 11 aa, Artificial Sequence
  LOS peptide mimotope sequence
CSHAPPYDRVC

SEQ ID NO: 155 - protein, 11 aa, Artificial Sequence
  LOS peptide mimotope sequence
CVTIPYRGTQC

SEQ ID NO: 156 - protein, 11 aa, Artificial Sequence
  LOS peptide mimotope sequence
CFAPPYDPLPC

SEQ ID NO: 157 - protein, 11 aa, Artificial Sequence
  LOS peptide mimotope sequence
CAPYSIFIGEC

SEQ ID NO: 158 - protein, 11 aa, Artificial Sequence
  LOS peptide mimotope sequence
CTHLYHYGTSC

SEQ ID NO: 159 - protein, 11 aa, Artificial Sequence
  LOS peptide mimotope sequence
CLCQAYKGRRC

SEQ ID NO: 160 - protein, 9 aa, Artificial Sequence
  LOS peptide mimotope sequence
CDPRLLDLC

SEQ ID NO: 161 - protein, 11 aa, Artificial Sequence
  LOS peptide mimotope sequence
CKTALPPYDRC

SEQ ID NO: 162 - protein, 11 aa, Artificial Sequence
  LOS peptide mimotope sequence
CFARPFQGTWC

SEQ ID NO: 163 - protein, 11 aa, Artificial Sequence
  LOS peptide mimotope sequence
CSLSLPPYDRC

SEQ ID NO: 164 - protein, 11 aa, Artificial Sequence
  LOS peptide mimotope sequence
CDRTLSALALC

SEQ ID NO: 165 - protein, 11 aa, Artificial Sequence
  LOS peptide mimotope sequence
CRAPPYDTIMC

SEQ ID NO: 166 - protein, 12 aa, Artificial Sequence
  LOS peptide mimotope sequence
CPPYDEGCRVAC

SEQ ID NO: 167 - protein, 11 aa, Artificial Sequence
  LOS peptide mimotope sequence
CFGLIAFHPDC

SEQ ID NO: 168 - protein, 11 aa, Artificial Sequence
  LOS peptide mimotope sequence
CQPIGPPYDRC

SEQ ID NO: 169 - protein, 11 aa, Artificial Sequence
  LOS peptide mimotope sequence
CTANYYFGTYC

SEQ ID NO: 170 - protein, 11 aa, Artificial Sequence
  LOS peptide mimotope sequence
CANSRPGGYLC

SEQ ID NO: 171 - protein, 11 aa, Artificial Sequence
  LOS peptide mimotope sequence
CMSSYGRGVRC

SEQ ID NO: 172 - protein, 11 aa, Artificial Sequence
  LOS peptide mimotope sequence
CVSTPFRGTFC

SEQ ID NO: 173 - protein, 11 aa, Artificial Sequence
  LOS peptide mimotope sequence
CDPRITPDFGC

SEQ ID NO: 174 - protein, 11 aa, Artificial Sequence
  LOS peptide mimotope sequence
CGPPYDPFPAC

SEQ ID NO: 175 - protein, 11 aa, Artificial Sequence
  LOS peptide mimotope sequence
CHTVRFRGTLC

SEQ ID NO: 176 - protein, 11 aa, Artificial Sequence
  LOS peptide mimotope sequence
CTAPPYDAYGC

SEQ ID NO: 177 - protein, 11 aa, Artificial Sequence
  LOS peptide mimotope sequence
CRSPLLGAPVC

SEQ ID NO: 178 - protein, 11 aa, Artificial Sequence
  LOS peptide mimotope sequence
CTTTYGTGTWC

SEQ ID NO: 179 - protein, 11 aa, Artificial Sequence
  LOS peptide mimotope sequence
CSSLYYHGTAC

SEQ ID NO: 180 - protein, 11 aa, Artificial Sequence
  LOS peptide mimotope sequence
CLYEPLRGTLC

SEQ ID NO: 181 - protein, 11 aa, Artificial Sequence
  LOS peptide mimotope sequence
CAPPPYDQSFC

SEQ ID NO: 182 - protein, 11 aa, Artificial Sequence
  LOS peptide mimotope sequence
CPPPWYSRSSC

SEQ ID NO: 183 - protein, 11 aa, Artificial Sequence
  LOS peptide mimotope sequence
CSRALGYVSEC

SEQ ID NO: 184 - protein, 11 aa, Artificial Sequence
  LOS peptide mimotope sequence
CPTWYKLKSVC

SEQ ID NO: 185 - protein, 11 aa, Artificial Sequence
  LOS peptide mimotope sequence
CRGSEGSFARC

SEQ ID NO: 186 - protein, 11 aa, Artificial Sequence
  LOS peptide mimotope sequence
CKQTIGSFDGC

SEQ ID NO: 187 - protein, 11 aa, Artificial Sequence
  LOS peptide mimotope sequence
CPLWYDPAPPC

SEQ ID NO: 188 - protein, 11 aa, Artificial Sequence
  LOS peptide mimotope sequence
CESPYSAHRWC

SEQ ID NO: 189 - protein, 11 aa, Artificial Sequence
  LOS peptide mimotope sequence
CWYDERTILKC

SEQ ID NO: 190 - protein, 11 aa, Artificial Sequence
  LOS peptide mimotope sequence
CSSYSYVHDSC

SEQ ID NO: 191 - protein, 11 aa, Artificial Sequence
  LOS peptide mimotope sequence
CRFTYDPPFMC

SEQ ID NO: 192 - protein, 11 aa, Artificial Sequence
  LOS peptide mimotope sequence
CRLYSFVFDKC

SEQ ID NO: 193 - protein, 11 aa, Artificial Sequence
  LOS peptide mimotope sequence
CSQWRSAAPTC

SEQ ID NO: 194 - protein, 11 aa, Artificial Sequence
  LOS peptide mimotope sequence
CRPAFDPPYHC

SEQ ID NO: 195 - protein, 11 aa, Artificial Sequence
  LOS peptide mimotope sequence
CHGRTLWYTPC

SEQ ID NO: 196 - protein, 11 aa, Artificial Sequence
  LOS peptide mimotope sequence
CSSVSATYPIC

SEQ ID NO: 197 - protein, 11 aa, Artificial Sequence
  LOS peptide mimotope sequence
CSLVQSPKRFC

SEQ ID NO: 198 - protein, 11 aa, Artificial Sequence
  LOS peptide mimotope sequence
CSNWYENTPTC

SEQ ID NO: 199 - protein, 11 aa, Artificial Sequence
  LOS peptide mimotope sequence
CPRPGWGQSAC

SEQ ID NO: 200 - protein, 12 aa, Artificial Sequence
  LOS peptide mimotope sequence
CTDPRGCGMFAC

SEQ ID NO: 201 - protein, 11 aa, Artificial Sequence
  LOS peptide mimotope sequence
CPRPHFGAPPC

SEQ ID NO: 202 - protein, 11 aa, Artificial Sequence
  LOS peptide mimotope sequence
CVTRATYPSWC

SEQ ID NO: 203 - protein, 11 aa, Artificial Sequence
  LOS peptide mimotope sequence
CWYIAPRKTLC

SEQ ID NO: 204 - protein, 11 aa, Artificial Sequence
  LOS peptide mimotope sequence
CYGYSALRDTC

SEQ ID NO: 205 - protein, 11 aa, Artificial Sequence
  LOS peptide mimotope sequence
CIITGSGWYVC

SEQ ID NO: 206 - protein, 11 aa, Artificial Sequence
  LOS peptide mimotope sequence
CTHYSFYGDIC

SEQ ID NO: 207 - protein, 11 aa, Artificial Sequence
  LOS peptide mimotope sequence
CHWYSTEAAWC

SEQ ID NO: 208 - protein, 11 aa, Artificial Sequence
  LOS peptide mimotope sequence
CHRIAQSLPQC

SEQ ID NO: 209 - protein, 11 aa, Artificial Sequence
  LOS peptide mimotope sequence
CALYRFAADSC

SEQ ID NO: 210 - protein, 11 aa, Artificial Sequence
  LOS peptide mimotope sequence
CRPQFDPPNDC

SEQ ID NO: 211 - protein, 11 aa, Artificial Sequence
  LOS peptide mimotope sequence
CHPALARWPLC

SEQ ID NO: 212 - protein, 11 aa, Artificial Sequence
  LOS peptide mimotope sequence
CQPKSLWYSVC

SEQ ID NO: 213 - protein, 11 aa, Artificial Sequence
  LOS peptide mimotope sequence
CRGYSHVSDAC

SEQ ID NO: 214 - protein, 11 aa, Artificial Sequence
  LOS peptide mimotope sequence
CDPVRTIYPIC

SEQ ID NO: 215 - protein, 11 aa, Artificial Sequence
  LOS peptide mimotope sequence
CWYTTPTRPVC

SEQ ID NO: 216 - protein, 11 aa, Artificial Sequence
  LOS peptide mimotope sequence
CQRQSLWYSSC

SEQ ID NO: 217 - protein, 11 aa, Artificial Sequence
  LOS peptide mimotope sequence
CNPDYSSPHEC

SEQ ID NO: 218 - protein, 11 aa, Artificial Sequence
  LOS peptide mimotope sequence
CPPWYPEHKTC

SEQ ID NO: 219 - protein, 11 aa, Artificial Sequence
  LOS peptide mimotope sequence
CASLGLAKTTC

SEQ ID NO: 220 - protein, 11 aa, Artificial Sequence
  LOS peptide mimotope sequence
CWYVDGPLATC

SEQ ID NO: 221 - protein, 11 aa, Artificial Sequence
  LOS peptide mimotope sequence
CRGWYADPSAC

SEQ ID NO: 222 - protein, 11 aa, Artificial Sequence
  LOS peptide mimotope sequence
CLWRPIDPFLC

SEQ ID NO: 223 - protein, 11 aa, Artificial Sequence
  LOS peptide mimotope sequence
CAERSLWYYPC

SEQ ID NO: 224 - protein, 11 aa, Artificial Sequence
  LOS peptide mimotope sequence
CPPWYNQSELC

SEQ ID NO: 225 - protein, 11 aa, Artificial Sequence
  LOS peptide mimotope sequence
CDSAPAVKSSC

SEQ ID NO: 226 - protein, 11 aa, Artificial Sequence
  LOS peptide mimotope sequence
CPGWYDAHPTC

SEQ ID NO: 227 - protein, 11 aa, Artificial Sequence
  LOS peptide mimotope sequence
CRGLQGHIAYC

SEQ ID NO: 228 - protein, 11 aa, Artificial Sequence
  LOS peptide mimotope sequence
CWYSAPENALC

SEQ ID NO: 229 - protein, 11 aa, Artificial Sequence
  LOS peptide mimotope sequence
CWYTAPSLSLC

SEQ ID NO: 230 - protein, 11 aa, Artificial Sequence
  LOS peptide mimotope sequence
CWYTNPSIAAC

SEQ ID NO: 231 - protein, 10 aa, Artificial Sequence
  LOS peptide mimotope sequence
CWYFSNELGC

SEQ ID NO: 232 - protein, 11 aa, Artificial Sequence
  LOS peptide mimotope sequence
CWYTLDIGPTC

SEQ ID NO: 233 - protein, 11 aa, Artificial Sequence
  LOS peptide mimotope sequence
CGPWHGPSSSC

SEQ ID NO: 234 - protein, 11 aa, Artificial Sequence
  LOS peptide mimotope sequence
CGDWPPFSAPC

SEQ ID NO: 235 - protein, 11 aa, Artificial Sequence
  LOS peptide mimotope sequence
CWYVGSVRSQC

SEQ ID NO: 236 - protein, 10 aa, Artificial Sequence
  LOS peptide mimotope sequence
CALDIAGGYC

SEQ ID NO: 237 - protein, 11 aa, Artificial Sequence
  LOS peptide mimotope sequence
CPPPSRGGYIC

SEQ ID NO: 238 - protein, 11 aa, Artificial Sequence
  LOS peptide mimotope sequence
CQAFDTSWTAC

SEQ ID NO: 239 - protein, 11 aa, Artificial Sequence
  LOS peptide mimotope sequence
CFLPCRRCGSC

SEQ ID NO: 240 - protein, 11 aa, Artificial Sequence
  LOS peptide mimotope sequence
CRPWQTAHFAC

SEQ ID NO: 241 - protein, 11 aa, Artificial Sequence
  LOS peptide mimotope sequence
CGQYSSSPFPC

SEQ ID NO: 242 - protein, 11 aa, Artificial Sequence
  LOS peptide mimotope sequence
CGRPGPYPADC

SEQ ID NO: 243 - protein, 11 aa, Artificial Sequence
  LOS peptide mimotope sequence
CTPLPDGGILC

SEQ ID NO: 244 - protein, 11 aa, Artificial Sequence
  LOS peptide mimotope sequence
CLKWGDGSSAC

SEQ ID NO: 245 - protein, 11 aa, Artificial Sequence
  LOS peptide mimotope sequence
CYPQLSHANPC

SEQ ID NO: 246 - protein, 11 aa, Artificial Sequence
  LOS peptide mimotope sequence
CSAYHRSLGAC

SEQ ID NO: 247 - protein, 11 aa, Artificial Sequence
  LOS peptide mimotope sequence
CFPLPSREFAC

SEQ ID NO: 248 - protein, 11 aa, Artificial Sequence
  LOS peptide mimotope sequence
CYRQSRSSWPC

SEQ ID NO: 249 - protein, 11 aa, Artificial Sequence
  LOS peptide mimotope sequence
CSHRFDALRRC

SEQ ID NO: 250 - protein, 11 aa, Artificial Sequence
  LOS peptide mimotope sequence
CVRFPDGSHSC

SEQ ID NO: 251 - protein, 11 aa, Artificial Sequence
  LOS peptide mimotope sequence
CSPAAFSDRLC

SEQ ID NO: 252 - protein, 11 aa, Artificial Sequence
  LOS peptide mimotope sequence
CVTDQWGGYLC

SEQ ID NO: 253 - protein, 11 aa, Artificial Sequence
  LOS peptide mimotope sequence
CVPSGRSPNTC

SEQ ID NO: 254 - protein, 11 aa, Artificial Sequence
  LOS peptide mimotope sequence
CPKWSDKRPQC

SEQ ID NO: 255 - protein, 11 aa, Artificial Sequence
  LOS peptide mimotope sequence
CAPPGIAVRTC

SEQ ID NO: 256 - protein, 11 aa, Artificial Sequence
  LOS peptide mimotope sequence
CMKWGPNSHSC

SEQ ID NO: 257 - protein, 11 aa, Artificial Sequence
  LOS peptide mimotope sequence
CLQDRAGGYLC

SEQ ID NO: 258 - protein, 12 aa, Artificial Sequence
  LOS peptide mimotope sequence
CGRLEGRCSHAC

SEQ ID NO: 259 - protein, 11 aa, Artificial Sequence
  LOS peptide mimotope sequence
CYFIAKHGWAC

SEQ ID NO: 260 - protein, 11 aa, Artificial Sequence
  LOS peptide mimotope sequence
CPPRSSRGFLC

SEQ ID NO: 261 - protein, 11 aa, Artificial Sequence
  LOS peptide mimotope sequence
CTGISTGEYLC

SEQ ID NO: 262 - protein, 11 aa, Artificial Sequence
  LOS peptide mimotope sequence
CGPVFYATGLC

SEQ ID NO: 263 - protein, 11 aa, Artificial Sequence
  LOS peptide mimotope sequence
CLSQYADWTYC

SEQ ID NO: 264 - protein, 11 aa, Artificial Sequence
  LOS peptide mimotope sequence
CARWYPISQTC

SEQ ID NO: 265 - protein, 11 aa, Artificial Sequence
  LOS peptide mimotope sequence
CQGFPGAPQDC

SEQ ID NO: 266 - protein, 11 aa, Artificial Sequence
  LOS peptide mimotope sequence
CWHFRTFPATC

SEQ ID NO: 267 - protein, 11 aa, Artificial Sequence
  LOS peptide mimotope sequence
CTRRPFDPPAC

SEQ ID NO: 268 - protein, 11 aa, Artificial Sequence
  LOS peptide mimotope sequence
CASPLGPCFWC

SEQ ID NO: 269 - protein, 11 aa, Artificial Sequence
  LOS peptide mimotope sequence
CWTDTYGDLLC

SEQ ID NO: 270 - protein, 11 aa, Artificial Sequence
  LOS peptide mimotope sequence
CISAGPESSHC

SEQ ID NO: 271 - protein, 11 aa, Artificial Sequence
  LOS peptide mimotope sequence
CHSVQPATRAC

SEQ ID NO: 272 - protein, 11 aa, Artificial Sequence
  LOS peptide mimotope sequence
CPKAPFSPFKC

SEQ ID NO: 273 - protein, 11 aa, Artificial Sequence
  LOS peptide mimotope sequence
CIDAGSHGWLC

SEQ ID NO: 274 - protein, 11 aa, Artificial Sequence
  LOS peptide mimotope sequence
CRRGSPLSRYC

SEQ ID NO: 275 - protein, 11 aa, Artificial Sequence
  LOS peptide mimotope sequence
CSWDEIIDLGC

SEQ ID NO: 276 - protein, 11 aa, Artificial Sequence
  LOS peptide mimotope sequence
CRSPAGEWSSC

SEQ ID NO: 277 - protein, 11 aa, Artificial Sequence
  LOS peptide mimotope sequence
CAYHVLRYSAC

SEQ ID NO: 278 - protein, 11 aa, Artificial Sequence
  LOS peptide mimotope sequence
CAKTVRGDYYC

SEQ ID NO: 279 - protein, 11 aa, Artificial Sequence
  LOS peptide mimotope sequence
CLAASADTAAC

SEQ ID NO: 280 - protein, 11 aa, Artificial Sequence
  LOS peptide mimotope sequence
CPYTSWAREGC

SEQ ID NO: 281 - DNA, 631 bp, Mouse
gacccagtttccacttttccctgcctgtcagtcttggagatcaaacctccatctcttgcacatctagtcggagccttgtacaccggtaaaggaaacacctatttacattggtaccttcagaagccaggccagtctccaaagctcctgatctacaaagtttccaaccgattttctggggtcccagacaggttcagtggcagtggatcagggacagatttcacactcacgatcagcagagtggaggctgaggatctgggactttatttctgctctcaaagtacacatgttccgtggacgttcggtggaggcaccaacctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcg

SEQ ID NO: 282 - DNA, 633 bp, Mouse
cctgagtctgaaggtggcctggtgcagcctgaaggatccctgaaactctcctgtgcagcctccggattcgattttagtagatactggatgagttgggtccggcaggctccagggaaagggctagaatggattggagaaatttatccagatagcagaacgataaactatacgccttctctaaaggataaattcatcatctccagagacaacgccaaaaatacgctatacctgcaaatgagcaaagtgagatctgaggacacaggcctttattactgtgcaatctactttacttacgacgcctgggttatggactactggggtcaaggaacctcagtcaccgtctcctcagctacaacaacagccccatctgtctatcccttggtccctggctgcagtgacacatctggatcctcggtgacactgggatgccttgtcaaaggctacttccctgagccggtaactgtaaaatggaactatggagccctgtccagcggtgtgcgcacagtctcatctgtcctgcagtctgggttctattccctcagcagcttggtgactgtaccctccagcacctggcccagccagactgtcatctgcaacgtagcccacccagccagcaagactgagttgtcaaga

SEQ ID NO: 283 - DNA, 625 bp, Mouse
cccagttccactttcctgcctgtcagtcttggagatcaagcctccatctcttgcagatctagtcagagccttgttcacaataagggaaacacctatttacattggtacctgcagaagccaggccagtctccaaagctcctgatctacaaagtttccaaccgattttctggggtccctgacaggttcagtggcagtgattcagggacagatttcacactcaggatcagcagagtggaggctgaggatctgggagtttatttctgctctcaaagtacacatgttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcg

SEQ ID NO: 284 - DNA, 625 bp, Mouse
  misc_feature 617: n = A,T,C or G
tctgaagtggctggtgcagctgaaggatccctgaaactctcctgtgtcgccacaggattcgattttagtagatactggatgagttgggtccggcaggctccagggaaagggctagagtggattggagaaatttatccagatagcaggaagataaactatacgccatctctaaaggataagttcatcatctccagagacaacgccaaaaatacgctgtacctgcaaatgagcaaagtgagatctgaggacacagccctttattactgtgcagtctactatacttacgacgtctgggctatggactactggggtcaaggaacctcagtcaccgtctcctcagctacaacaacagccccatctgtctatcccttggtccctggctgcagtgacacatctggatcctcggtgacactgggatgccttgtcaaaggctacttccctgagccggtaactgtaaaatggaactatggagccctgtccagcggtgtgcgcacagtctcatctgtcctgcagtctgggttctattccctcagcagcttggtgactgtaccctccagcacctggcccagccagactgtcatctgcaacgtagcccacccagccagcaagactgagtnatcaagag

SEQ ID NO: 285 - DNA, 632 bp, Mouse
tgacccagtctccactcacttttgtcggttaccattggacaaccagcctccatctcttgcaagtcaagtcagagcctcttagataatgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcgg

SEQ ID NO: 286 - DNA, 619 bp, Mouse
acaggtaagctggggcttcagtgaggatatcctgtaaggcttctggctacacctttcacaagctactatatacactgggtgaagcagaggcctggacagggacttgagtggattggatggatttatcctggaaatgttaatactaagtacaatgagaagttcaagggcaaggccacactgactgcagacaaatcctccagcacagcctacatgcagctcagcagcctgacctctgaggactctgcggtctatttctgtgcaagagggggggtacggtatgctatggactactggggtcaaggaacctcagtcaccgtctcctcagagagtcagtccttcccaaatgtcttccccctcgtctcctgcgagagccccctgtctgataagaatctggtggccatgggctgcctggcccgggacttcctgcccagcaccatttccttcacctggaactaccagaacaacactgaagtcatccagggtatcagaaccttcccaacactgaggacagggggcaagtacctagccacctcgcaggtgttgctgtctcccaagagcatccttgaaggttcagatgaatacctggtatgcaaaatccactacggaggcaaaaacagag

SEQ ID NO: 287 - DNA, 630 bp, Mouse
ctgacccagttccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcg

SEQ ID NO: 288 - DNA, 634 bp, Mouse
gtctggacctaagtggtaaagcctggggcttcagtgaggatatcctgcaaggcttctggctacaccttcacaagctactatatacactgggtgaagcagaggcctggacagggacttgagtggattggatggatttatcctggaaatgttaatactaagtacaatgagaagttcaagggcaaggccacactgactgcagacaaatcctccagcacagcctacatgcagctcagcagcctgacctctgaggactctgcggtctatttctgtgcaagatctactacggctagggggtactttgactactggggccaaggcaccactctcacagtctcctcagagagtcagtccttcccaaatgtcttccccctcgtctcctgcgagagccccctgtctgataagaatctggtggccatgggctgcctggcccgggacttcctgcccagcaccatttccttcacctggaactaccagaacaacactgaagtcatccagggtatcagaaccttcccaacactgaggacagggggcaagtacctagccacctcgcaggtgttgctgtctcccaagagcatccttgaaggttcagatgaatacctggtatgcaaaatccactacggaggcaaaaacagag

SEQ ID NO: 289 - protein, 8 aa, Artificial Sequence
  LOS peptide mimotope sequence
PPPWSTHD

SEQ ID NO: 290 - protein, 8 aa, Artificial Sequence
  LOS peptide mimotope sequence
SEPWSTSN

SEQ ID NO: 291 - protein, 9 aa, Artificial Sequence
  LOS peptide mimotope sequence
AITGVRARW

SEQ ID NO: 292 - protein, 9 aa, Artificial Sequence
  LOS peptide mimotope sequence
EKKHFNYGT

SEQ ID NO: 293 - protein, 9 aa, Artificial Sequence
  LOS peptide mimotope sequence
EKKRFESNT

SEQ ID NO: 294 - protein, 16 aa, Artificial Sequence
  LOS peptide mimotope sequence
EQGYCTVNIEQCAKYR

SEQ ID NO: 295 - protein, 15 aa, Artificial Sequence
  LOS peptide mimotope sequence
SPADCDYTTLCAKPT

SEQ ID NO: 296 - protein, 14 aa, Artificial Sequence
  LOS peptide mimotope sequence
NSPTSCKWLCNEKF

SEQ ID NO: 297 - protein, 10 aa, Artificial Sequence
  LOS peptide mimotope sequence
CPPPWSTHDC

SEQ ID NO: 298 - protein, 10 aa, Artificial Sequence
  LOS peptide mimotope sequence
CSEPWSTSNC

SEQ ID NO: 299 - protein, 11 aa, Artificial Sequence
  LOS peptide mimotope sequence
CAITGVRARWC

SEQ ID NO: 300 - protein, 11 aa, Artificial Sequence
  LOS peptide mimotope sequence
CEKKHFNYGTC

SEQ ID NO: 301 - protein, 11 aa, Artificial Sequence
  LOS peptide mimotope sequence
CEKKRFESNTC

SEQ ID NO: 302 - protein, 7 aa, Artificial Sequence
  LOS peptide mimotope sequence
IHRQGIH

SEQ ID NO: 303 - protein, 7 aa, Artificial Sequence
  LOS peptide mimotope sequence
HIGQRHI

SEQ ID NO: 304 - protein, 7 aa, Artificial Sequence
  LOS peptide mimotope sequence
LPARTEG

SEQ ID NO: 305 - protein, 7 aa, Artificial Sequence
  LOS peptide mimotope sequence
GETRAPL

SEQ ID NO: 306 - protein, 7 aa, Artificial Sequence
  LOS peptide mimotope sequence
APARQLP

SEQ ID NO: 307 - protein, 7 aa, Artificial Sequence
  LOS peptide mimotope sequence
PLQRAPA

SEQ ID NO: 308 - protein, 7 aa, Artificial Sequence
  LOS peptide mimotope sequence
KQAPVHH

SEQ ID NO: 309 - protein, 7 aa, Artificial Sequence
  LOS peptide mimotope sequence
HHVPAQK

SEQ ID NO: 310 - protein, 7 aa, Artificial Sequence
  LOS peptide mimotope sequence
LQAPVHH

SEQ ID NO: 311 - protein, 7 aa, Artificial Sequence
  LOS peptide mimotope sequence
HHVPAQL

SEQ ID NO: 312 - protein, 7 aa, Artificial Sequence
  LOS peptide mimotope sequence
LPSIQLP

SEQ ID NO: 313 - protein, 7 aa, Artificial Sequence
  LOS peptide mimotope sequence
PLQISPL

SEQ ID NO: 314 - protein, 7 aa, Artificial Sequence
  LOS peptide mimotope sequence
NELPHKL

SEQ ID NO: 315 - protein, 7 aa, Artificial Sequence
  LOS peptide mimotope sequence
LKHPLEN

SEQ ID NO: 316 - protein, 7 aa, Artificial Sequence
  LOS peptide mimotope sequence
KSPSMTL

SEQ ID NO: 317 - protein, 7 aa, Artificial Sequence
  LOS peptide mimotope sequence
LTMSPSK

SEQ ID NO: 318 - protein, 7 aa, Artificial Sequence
  LOS peptide mimotope sequence
AGPLMLL

SEQ ID NO: 319 - protein, 7 aa, Artificial Sequence
  LOS peptide mimotope sequence
LLMLPGA

SEQ ID NO: 320 - protein, 7 aa, Artificial Sequence
  LOS peptide mimotope sequence
WSPILLD

SEQ ID NO: 321 - protein, 7 aa, Artificial Sequence
  LOS peptide mimotope sequence
DLLIPSW

SEQ ID NO: 322 - protein, 7 aa, Artificial Sequence
  LOS peptide mimotope sequence
LSMHPQN

SEQ ID NO: 323 - protein, 7 aa, Artificial Sequence
  LOS peptide mimotope sequence
NQPHMSL

SEQ ID NO: 324 - protein, 7 aa, Artificial Sequence
  LOS peptide mimotope sequence
HSMHPQN

SEQ ID NO: 325 - protein, 7 aa, Artificial Sequence
  LOS peptide mimotope sequence
NQPHMSH

SEQ ID NO: 326 - protein, 7 aa, Artificial Sequence
  LOS peptide mimotope sequence
SMYGSYN

SEQ ID NO: 327 - protein, 7 aa, Artificial Sequence
  LOS peptide mimotope sequence
NYSGYMS

SEQ ID NO: 328 - protein, 7 aa, Artificial Sequence
  LOS peptide mimotope sequence
TNHSLYH

SEQ ID NO: 329 - protein, 7 aa, Artificial Sequence
  LOS peptide mimotope sequence
HYLSHNT

SEQ ID NO: 330 - protein, 7 aa, Artificial Sequence
  LOS peptide mimotope sequence
HAIYPRH

SEQ ID NO: 331 - protein, 7 aa, Artificial Sequence
  LOS peptide mimotope sequence
HRPYIAH

SEQ ID NO: 332 - protein, 7 aa, Artificial Sequence
  LOS peptide mimotope sequence
TTYSRFP

SEQ ID NO: 333 - protein, 7 aa, Artificial Sequence
  LOS peptide mimotope sequence
PFRSYTT

SEQ ID NO: 334 - protein, 7 aa, Artificial Sequence
  LOS peptide mimotope sequence
TDSLRLL

SEQ ID NO: 335 - protein, 7 aa, Artificial Sequence
  LOS peptide mimotope sequence
LLRLSDT

SEQ ID NO: 336 - protein, 7 aa, Artificial Sequence
  LOS peptide mimotope sequence
SFATNIP

SEQ ID NO: 337 - protein, 7 aa, Artificial Sequence
  LOS peptide mimotope sequence
PINTAFS

SEQ ID NO: 338 - DNA, 34 bp, Artificial Sequence
  Primer
gcgccgtctagaattaacactcattcctgttgaa

SEQ ID NO: 339 - DNA, 32 bp, Artificial Sequence
  Primer
ccagttccgagctcgttgtgactcaggaatct

SEQ ID NO: 340 - DNA, 32 bp, Artificial Sequence
  Primer
ccagttccgagctcgtgttgacgcagccgccc

SEQ ID NO: 341 - DNA, 32 bp, Artificial Sequence
  Primer
ccagttccgagctcgtgctcacccagtctcca

SEQ ID NO: 342 - DNA, 32 bp, Artificial Sequence
  Primer
ccagttccgagctcgagatgacacagtctcca

SEQ ID NO: 343 - DNA, 32 bp, Artificial Sequence
  Primer
ccagatgtgagctcgtgatgacccagactcca

SEQ ID NO: 344 - DNA, 22 bp, Artificial Sequence
  Primer
aggtccaactgctcgagtcagg

SEQ ID NO: 345 - DNA, 31 bp, Artificial Sequence
  Primer
ggggggtactagtcttgggtattctaggctc

SEQ ID NO: 346 - skipped
  Primer

SEQ ID NO: 347 - DNA, 17 bp, Artificial Sequence
  Primer
attctagagattacgcc

SEQ ID NO: 348 - DNA, 20 bp, Artificial Sequence
  Primer
cccatccccctgttgacaat

SEQ ID NO: 349 - DNA, 20 bp, Artificial Sequence
  Primer
tgctgcaaggcgattaagtt

SEQ ID NO: 350 - DNA, 20 bp, Artificial Sequence
  Primer
attaggcgggctgggtatct

SEQ ID NO: 351 - DNA, 17 bp, Artificial Sequence
  Primer
gttttcccagtcacgac

SEQ ID NO: 352 - DNA, 18 bp, Artificial Sequence
  Primer
catcggctcgtataatgt

(19) United States
(12) Patent Application Publication
(10) Pub. No.: US 2004/0047880 A1
(43) Pub. Date: Mar. 11, 2004

(54) Component for vaccine

(76) Inventors: Xavier Thomas De Bolle, Namur (BE); Jean-Jacques Letesson, Namur (BE); Yves Lobet, Rixensart (BE); Pascal Yvon Mertens, Namur (BE); Jan Poolman, Rixensart (BE); Pierre Voet, Rixensart (BE)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/398,104

(22) PCT Filed: Oct. 3, 2001
(86) PCT No.: PCT/EP01/11409

(30) Foreign Application Priority Data
Oct. 3, 2000 (GB) 0024200.8

(51) Int. Cl. A61K 39/02; A61K 31/739

(52) U.S. Cl. 424/190.1; 514/54

(57) ABSTRACT

The present invention relates to a component for a vaccine against menigococci, in particular peptides which mimic epitopes of meningococcal lipooligosaccharide, and to a vaccine comprising such a component.

### FIELD OF THE INVENTION

The present invention relates to a component for a vaccine against meningococci, preferably peptides which mimic epitopes of meningococcal lipooligosaccharide, and to a vaccine comprising such a component.

### BACKGROUND TO THE INVENTION

Neisseria meningitidis (meningococcus) is a Gram negative bacterium frequently isolated from the human upper respiratory tract. It is a cause of serious invasive bacterial diseases such as bacteremia and meningitis. The incidence of meningococcal disease shows geographical, seasonal and annual differences (Schwartz, B., Moore, P. S., Broome, C. V.; Clin. Microbiol. Rev. 2 (Supplement), S18-S24, 1989). The bacterium is commonly classified according to the serogroup if its capsular polysaccharide.

Most disease in temperate countries is due to strains of serogroup B and varies in incidence from 1-10/100,000/year total population—sometimes reaching higher values (Kaczmarski, E. B. (1997), Commun. Dis. Rep. Rev. 7: R55-9, 1995; Scholten, R. J. P. M., Bijlmer, H. A., Poolman, J. T. et al. Clin. Infect. Dis. 16: 237-246, 1993; Cruz, C., Pavez, G., Aguilar, E., et al. Epidemiol. Infect. 105: 119-126, 1990).

Epidemics dominated by serogroup A meningococci, mostly in central Africa, sometimes reach incidence levels of up to 1000/100,000/year (Schwartz, B., Moore, P. S., Broome, C. V. Clin. Microbiol. Rev. 2 (Supplement), S18-S24, 1989). Nearly all cases as a whole of meningococcal disease are caused by serogroup A, B, C, W-135 and Y meningococci, and a tetravalent A, C, W-135, Y capsular polysaccharide vaccine is available (Armand, J., Arminjon, F., Mynard, M. C., Lafaix, C., J. Biol. Stand. 10: 335-339, 1982).

The frequency of Neisseria meningitidis infections has risen dramatically in the past few decades. This has been attributed to the emergence of multiple antibiotic resistant stains and an increasing population of people with weakened immune systems. It is no longer uncommon to isolate Neisseria meningitidis strains that are resistant to some or all of the standard antibiotics. This phenomenon has created an unmet medical need and demand for new anti-microbial agents, vaccines, drug screening methods, and diagnostic tests for this organism.

The available polysaccharide vaccines are currently being improved by way of chemically conjugating them to carrier proteins (Lieberman, J. M., Chiu, S. S., Wong, V. K., et al. JAMA 275: 1499-1503, 1996).

A serogroup B vaccine, however, is not available. The serogroup B capsular polysaccharide has been found to be nonimmunogenic—most likely because it shares structural similarity with host components (Wyle, F. A., Artenstein, M. S., Brandt, M. L. et al. J. Infect. Dis. 126: 514-522, 1972; Finne, J. M., Leinonen, M., Mäkelä, P. M. Lancet ii.: 355-357, 1983). Effort has therefore been focused in trying to develop serotype B vaccines from outer membrane vesicles or purified protein components therefrom.

Alternative meningococcal antigens for vaccine development are meningococcal lipooligosaccharides (LOS). These are outer membrane bound glycolipids which differ from the lipopolysaccharides (LPS) of the Enterobacteriaceae by lacking the O side chains, and thus resemble the rough form of LPS (Griffiss et al. Rev Infect Dis 1988; 10: S287-295). Heterogeneity within the oligosaccharide moiety of the LOS generates structural and antigenic diversity among different meningococcal strains (Griffiss et al. Inf. Immun. 1987; 55: 1792-1800). This has been used to subdivide the strains into 12 immunotypes. Immunotypes L3, L7, L9 have an identical carbohydrate structure and have therefore been designated L3,7,9. Meningococcal LOS L3,7,9, L2 and L5 can be modified by sialylation, or by the addition of cytidine 5′-monophosphate-N-acetylneuraminic acid. Antibodies to LOS have been shown to protect in experimental rats against infection and to contribute to the bactericidal activity in children infected with N. meningitidis (Griffiss et al J Infect Dis 1984; 150: 71-79).

The toxic component of the LOS, as in the case of endotoxin from other Gram-negative bacteria, lies in the lipid A moiety of the molecule, and not in the immunogenic oligosaccharide portion. Although it may be possible to separate the toxic part from the immunogenic portion of the molecule, once done the native conformation of the molecule may not be retained.

A solution to this difficulty is the identification of mimotopes which can mimic epitopes on the oligosaccharide moiety of the LOS. In this way surrogate antigens may be generated.

Peptides mimicking polysaccharides have been reported. For instance, mimotopes of meningococcal group B capsular polysaccharide (Moe et al. 1999. FEMS Immunology and Medical Microbiology 26: 209-226) and meningococcal group C capsular polysaccharide (Westerink et al. 1995 Proc. Natl. Acad. Sci. USA 92: 4021-4025) have been identified. Furthermore, WO 00/25814 discloses several serogroup B LOS L3,7,9 heptapeptide mimotopes.

As mimotopes can vary widely in their suitability for inclusion in a vaccine (that is whether they constitute an immunogenic mimotope which can induce a protective humoral immune response against the carbohydrate), there remains a need to identify further classes of peptide mimotopes of meningococcal (particularly serogroup B) LOS.

### SUMMARY OF THE INVENTION

To achieve the identification of further peptide mimotopes mimicking surface-exposed epitopes of N. meningitidis LOS, the present inventors have screened two phage-displayed random peptide libraries with five monoclonal antibodies directed against N. meningitidis LOS.

The present invention provides a mimotope of a surface L3,7,9 LOS of N. meningitidis, said mimotope being antigenically cross-reactive with a monoclonal antibody selected from a group consisting of: 4BE12C10; H44/24; H44/58; H44/70; and H44/78.

The present invention further provides a mimotope of a surface L3,7,9 LOS of N. meningitidis, said mimotope comprising a peptide epitope obtainable by screening a peptide library with a monoclonal antibody selected from a group consisting of: 4BE12C10; H44/24; H44/58; H44/70; and H44/78.

Vaccine compositions comprising the above mimotopes are also provided.

A further aspect of the invention relates to a vaccine against serogroup B, C, Y, or W-135 meningococci, which comprises a mimotope of a surface L3,7,9 LOS of N. meningitidis and a mimotope of a surface L2 LOS of N. meningitidis.

A still further aspect relates to a vaccine against serogroup A meningococci, which comprises a mimotope of a surface L3,7,9 LOS of N. meningitidis and a mimotope of a surface L10 LOS of N. meningitidis.

### DETAILED DESCRIPTION OF THE INVENTION

The present invention aims to provide further classes of N. meningitidis LOS mimotopes for use as a component of a meningococcal vaccine.

The present invention provides a mimotope of a surface L3,7,9 LOS of N. meningitidis, said mimotope being antigenically cross-reactive with a monoclonal antibody selected from a group consisting of: 4BE12C10; H44/24; H44/58; H44/70; and H44/78. These monoclonal antibodies are each described later and are termed the ‘mAbs of the invention’. These mAbs have high specificity and/or affinity to the L3,7,9 LOS. The meaning of mimotope is defined as an entity which is sufficiently similar to a native meningococcal LOS epitope so as to be capable of being bound by antibodies which recognise the native meningococcal LOS epitope. ‘Antigenically cross-reactive’ for the purposes of this invention means that the mimotope tests positive in an ELISA test (preferably as performed in Example 3) or immunoblot on recombinant phages expressing the mimotope. Preferably, the mimotope does not have a naturally-occurring amino-acid sequence.

Preferably, the mimotopes of the invention can be used to immunise a host such that antibodies are produced which specifically cross-react with LOS, and preferably cross-react with whole cell bacteria containing the LOS.

The present invention further provides a mimotope of a surface L3,7,9 LOS of N. meningitidis, said mimotope comprising a peptide epitope obtainable by screening a peptide library with a monoclonal antibody selected from a group consisting of: 4BE12C10; H44/24; H44/58; H44/70; and H44/78. Preferably, the mimotope does not have a naturally-occurring amino-acid sequence.

Typically, the peptide epitope is obtainable by screening a peptide library (preferably a random, highly diverse one) with a monoclonal antibody of high specificity and/or affinity to the LOS. Typically techniques such as phage display technology (EP 0 552 267 B1) can be used for screening such libraries (preferably as described in Example 2). This technique has the advantageous potential of allowing the identification of many peptide mimotopes so that a recognition pattern can be established in order to define essential features (or chemical properties) of an epitope contained within a peptide mimotope of L3,7,9 LOS.

In the present invention, a nonamer peptide library (either linear or disulfide-constrained, for instance as previously described by Felici et al. [1993 Gene 128: 21-27] and Luzzago et al. [1993 Gene 128: 51-57]) was found to be conveniently used to challenge the mAbs of the invention in order to identify peptide epitopes contained within the peptide mimotopes of SEQ ID NO: 1-140, 289-296 that were isolated from the libraries. Thus the mimotope of the invention preferably comprises a peptide epitope contained within any one of the peptides of SEQ ID NO: 1-140, 289-296, or retro sequences thereof.

According to the present invention, the peptide epitope may comprise a sub-sequence of any one of SEQ ID NO:1-140, 289-296, or retro-sequences thereof, or may be present in a longer peptide incorporating any one of SEQ ID NO:1-140, 289-296 (or retro-sequences thereof) or sub-sequences therefrom. Accordingly, the mimotopes of the present invention may consist of addition of N and/or C terminal extensions of a number of other natural residues at one or both ends of the peptides of SEQ ID NO:1-140, 289-296.

Preferably the mimotope of the invention comprises any one of the peptides of SEQ ID NO: 1-140, 289-296 (the peptides of the invention), or retro sequences thereof, or modifications of the peptides or retro sequences. Most preferably, the mimotopes comprising the retro sequences and modifications of the peptides of the invention should retain cross-reactivity with a monoclonal antibody selected from a group consisting of: 4BE12C10; H44/24; H44/58; H44/70; and H44/78. Most preferably, the mimotope of the invention comprises any one of the peptides of SEQ ID NO:153, 154, 157, 162, 167, 168, 169, 170, 179, 45, 47, 190, 191, 53, 194, 55, 58, 61, 63, 206, 75, 222, 83, 85, 86, 227, 88, 93, 243, 104, 245, 255, 124, 271, 272, 273, 279, 280, 297, 298, 291, 292, 293, 294, 295, and 296 (corresponding, respectively, to peptide No. 13, 14, 17, 22, 27, 28, 29, 30, 39, 45, 47, 50, 51, 53, 54, 55, 58, 61, 63, 66, 75, 82, 83, 85, 86, 87, 88, 93, 103, 104, 105, 115, 124, 131, 132, 133, 139, 140, 141, 142, 143, 144, 145, 146, 147, and 148 with reference to Table 2 in Example 2) or retro sequences and modifications of the peptides which are still recognised by one or more mAbs in the ELISA test of Example 3.

By ‘retro sequences’ with reference to a peptide sequence it is meant peptide sequences where the sequence orientation is reversed. Thus a retro sequence of the peptide AGDT is TDGA. It has been found in the art that retro sequences of peptide mimotopes are often peptide mimotopes themselves. Peptide mimotope sequences of the invention may be entirely or at least in part comprised of D-stereo isomer amino acids (inverso sequences). Also, the peptide sequences may be retro-inverso in character, in that the sequence orientation is reversed and the amino acids are of the D-stereoisomer form. Such retro, inverso or retro-inverso peptides have the advantage of potentially being more stable and/or immunogenic in a host when administered as an immunogen. Methods to make D amino acids and incorporate them into proteins are well known in the art [see, for example, Thorson et al. (1998) Methods Mol. Biol. 77:43-73, & Chartrain et al. (2000) Curr. Opin. Biotechnol. 11:209-14].

Peptide mimotopes comprising the peptides of the invention may be modified (modifications of the peptides of the invention) for a particular purpose by addition, deletion or substitution of elected amino acids.

For example, 1, 2, 3, 4, 5, 6, 7, 8 or 9 amino acids of each of the peptides of SEQ ID NO:1-140, 289-296 can be replaced by the amino acid that most closely resembles that amino acid. For example, A may be substituted by V, L or I, as described in the following table.

| | Exemplary | Preferred |
|---|---|---|
| Original residue | substitutions | substitution |
| A | V, L, I | V |
| R | K, Q, N | K |
| N | Q, H, K, R | Q |
| D | E | E |
| C | S | S |
| Q | N | N |
| E | D | D |
| G | P, A | A |
| H | N, Q, K, R | R |
| I | L, V, M, A, F | L |
| L | I, V, M, A, F | I |
| K | R, Q, N | R |
| M | L, F, I | L |
| F | L, V, I, A, Y | L |
| P | A | A |
| S | T | T |
| T | S | S |
| W | Y, F | Y |
| Y | W, F, T, S | F |
| V | I, L, M, F, A | L |

Furthermore, the peptides of the present invention may be modified for the purposes of ease of conjugation to a protein carrier. For example, it may be desirable for some chemical conjugation methods to include a terminal cysteine to the peptide. In addition it may be desirable for peptides conjugated to a protein carrier to include a hydrophobic terminus distal from the conjugated terminus of the peptide, such that the free unconjugated end of the peptide remains associated with the surface of the carrier protein. This reduces the conformational degrees of freedom of the peptide, and thus increases the probability that the peptide is presented in a conformation which most closely resembles that of the L3,7,9 LOS epitope in the context of the whole molecule. For example, the peptides may be modified to have an N-terminal cysteine and a C-terminal hydrophobic amidated tail. Alternatively, the addition or substitution of a D-stereoisomer form of one or more of the amino acids may be performed to create a beneficial derivative, for example to enhance stability of the peptide. Those skilled in the art will realise that such modified peptides, or mimotopes, could be a wholly or partly non-peptide mimotope wherein the constituent residues are not necessarily confined to the 20 naturally occurring amino acids. Furthermore, one or more amino acids may be deleted from the peptides of the invention, as long as an epitope is retained which is capable of cross-reacting with the monoclonal antibodies of the invention. Typically such an epitope would have at least 4, 5, 6, 7 or 8 amino acids. In addition, the peptides of the invention may be cyclised by techniques known in the art to constrain the peptide into a conformation that closely resembles its shape when the peptide sequence is in the context of the whole LOS molecule.

Thus in a preferred further embodiment, the mimotope may comprise an oligopeptide which is structurally more constrained than a linear form of the oligopeptide. It is thought that peptides which assume fewer conformations or which have their conformations locked are more likely to elicit an immune response because they present to the binding portion of antibodies a structurally constrained epitope.

Substituents such as covalent linkages to further peptide chains or intramolecular linkages will structurally constrain the oligopeptide. For example, the oligopeptide may form part of the primary structure of a larger polypeptide containing the amino acid sequence of the oligopeptide. Preferably, the oligopeptide comprises a cyclic peptide, as discussed in further detail below.

Other substituents include covalent linkages to other moieties such as macromolecular structures including biological and non-biological structures. Examples of biological structures include carrier proteins such as those described below for enhancing the immunogenicity of the mimotope. Examples of non-biological structures include lipid vesicles such as micelles and the like.

In a preferred embodiment, the oligopeptide comprises a cyclic peptide. Use of a cyclic peptide. Typically, the cyclic peptide comprises a cyclised portion, which cyclised portion preferably comprises an amino acid sequence, the terminal amino acids of which are linked together by a covalent bond. The covalent bond is conveniently a disulphide bridge, such as found between cysteine residues. The cyclised portion typically comprises a nonapeptide and this nonapeptide can conveniently form part of the amino acid sequence which is flanked by the amino acids which are linked by the covalent bond to form the cyclised portion.

Examples of preferred cyclised peptides which contain a pair of cysteine residues to allow the formation of a disulphide bridge are SEQ ID NO:141-280, 297-301 (and retro, inverso, or retroinverso variants thereof, as defined above).

As described above, the large number of peptide mimotopes identified by the phage display technique allows the identification of patterns which define an epitope (or part of an epitope) of a mimotope of L3,7,9 LOS. Accordingly a further aspect of the invention is peptide mimotopes of L3,7,9 LOS comprising the amino acid sequence (either linear or cyclised): WY; PP; AP; PY; PPY; PPF; PPW; APP; WYS; WYT; LWY; GGY; GPY; PPYD (a preferred motif); PPFD; FDPP; GGYL; PPWD; SLWY; PXWY; WYXXP; YXY; PWST; EKKXF or WXY (where each X is the same or different and is an amino acid, preferably a naturally-occurring amino acid).

In a preferred embodiment, the peptides incorporating the above identified epitopes or peptidic mimotopes of the present invention will be of a small size. It is envisaged that peptidic mimotopes, therefore, should be less than 100 amino acids in length, preferably shorter than 75 amino acids, more preferably less than 50 amino acids, and most preferable within the range of 4 to 25 amino acids long. In a specific embodiment the peptide is 9 amino acids in length.

It will be apparent to the man skilled in the art that techniques may be used to confirm the status of a specific construct as a mimotope suitable for use in a vaccine against meningococcus. Such techniques include the following: the putative mimotope can be assayed to ascertain the immunogenicity of the construct, in that antisera raised by the putative mimotope cross-react with the native L3,7,9 LOS molecule, and are also functional in bactericidal assays against N. meningiditis of the L3,7,9 immunotype. Typically bactericidal assays are performed as described in Example 1.4. They may also be done using standard opsonophagocytosis experiments in an animal model such as the infant rat.

In one embodiment of the present invention at least one peptide as hereinbefore described, incorporating a peptide epitope or mimotope, is linked to carrier molecules to form immunogens for vaccination protocols. The peptides may be linked via chemical covalent conjugation or by expression of genetically engineered fusion partners, optionally via a linker sequence.

The covalent coupling of the peptide to the immunogenic carrier can be carried out in a manner well known in the art. Thus, for example, for direct covalent coupling it is possible to utilise a carbodiimide, glutaraldehyde or (N-[γ-maleimidobutyryloxy]) succinimide ester, utilising common commercially available heterobifunctional linkers such as CDAP and SPDP (using manufacturers instructions). After the coupling reaction, the immunogen can easily be isolated and purified by means of a dialysis method, a gel filtration method, a fractionation method etc.

The types of carriers used in the immunogens of the present invention will be readily known to the man skilled in the art The function of the carrier is to provide cytoline help in order to help induce an immune response against the peptide of the invention. A non-exhaustive list of carriers which may be used in the present invention include: Keyhole limpet Haemocyanin (KLH), serum albumins such as bovine serum albumin (BSA), inactivated bacterial toxins such as tetanus or diptheria toxins (TT and DT), or recombinant fragments thereof (for example, Domain 1 of Fragment C of TT, or the translocation domain of DT), CRM197, or the purified protein derivative of tuberculin (PPD). Alternatively the mimotopes or epitopes may be directly conjugated to liposome carriers, which may additionally comprise immunogens capable of providing T-cell help. Preferably the ratio of peptides to carrier is in the order of 1:1 to 20:1, and-preferably each carrier should carry between 3-15 peptides.

In an embodiment of the invention a preferred carrier is Protein D (an IgD-binding protein) from Haemophilus influenzae (WO 91/18926, EP 0 594 610 B1). In some circumstances, for example in recombinant immunogen expression systems it may be desirable to use fragments of protein D, for example Protein D ⅓^{rd} (comprising the N-terminal 100-110 amino acids of protein D (WO 99/10375)).

Another preferred method of presenting the peptides of the present invention is in the context of a recombinant fusion molecule. For example, EP 0 421 635 B describes the use of chimeric hepadnavirus core antigen particles to present foreign peptide sequences in a virus-like particle. As such, immunogens of the present invention may comprise peptides presented in chimeric particles consisting of hepatitis B core antigen. Additionally, the recombinant fusion proteins may comprise the mimotopes of the present invention and a carrier protein, such as NS1 of the influenza virus.

Alternatively, the peptides of the present invention could be inserted within or substitute a surface-exposed loop of an outer membrane protein (preferably of meningococcal origin, for example PorA, PorB, PilC, ThpA, FrpB or LbpA). This has the advantage of constraining the peptide into a shape that can mimic the LOS epitope. Additionally, this may be advantageous in terms of administering the immunogen to a host in an outer membrane vesicle preparation (or bleb preparation) from a meningococcal strain expressing the immunogen. Such an improved bleb preparation is a further aspect of the invention.

For any recombinantly expressed protein which forms part of the present invention, a nucleic acid sequence which encodes said immunogen also forms an aspect of the present invention. Additionally, DNA sequences encoding any aforementioned peptide or mimotope of the present invention are further aspects.

DNA molecules, for instance plasmids, comprising the DNA sequences of the present invention may be used as an immunogen in the manner described by Kieber-Emmons et al. (Journal of Immunology 2000 165:623-627). Such a strategy may advantageously trigger a cross-reactive Th1 immune response against the LOS in the host. A vaccine comprising such DNA molecules, and the use of such a vaccine for the treatment or prevention of meningococcal disease are further aspects of the invention.

Peptides used in the present invention can be readily synthesised by solid phase procedures well known in the art. Suitable syntheses may be performed by utilising “T-boc” or “F-moc” procedures. Cyclic peptides can be synthesised by the solid phase procedure employing the well-known “F-moc” procedure and polyamide resin in the fully automated apparatus. Alternatively, those skilled in the art will know the necessary laboratory procedures to perform the process manually. Techniques and procedures for solid phase synthesis are described in ‘Solid Phase Peptide Synthesis: A Practical Approach’ by E. Atherton and R. C. Sheppard, published by IRL at Oxford University Press (1989). Alternatively, the peptides may be produced by recombinant methods, including expressing nucleic acid molecules encoding the mimotopes in a bacterial or mammalian cell line, followed by purification of the expressed mimotope. Techniques for recombinant expression of peptides and proteins are known in the art, and are described in Maniatis, T., Fritsch, E. F. and Sambrook et al., Molecular cloning, a laboratory manual, 2nd Ed.; Cold Spring Harbor Laboratory Press, Cold Spring Harbor, N.Y. (1989).

The monoclonal antibodies of the invention, and pharmaceutical compositions comprising them, form part of the present invention. These antibodies (H44/24, H44/58, H44/70, H44/78 and 4BE12C10) are capable of being used in passive prophylaxis or therapy, by administration of the antibodies into a patient, for the amelioration or prevention of meningococcal disease. These antibodies are preferably made from a hybridoma The H44/24, H44/58, H44/70 and H44/78 hybridomas of the invention have been deposited as Budapest Treaty patent deposit at ECACC (European Collection of Cell Cultures, Vaccine Research and Production Laboratory, Public Health Laboratory Service, Centre for Applied Microbiology Research, Porton Down, Salisbury, Wiltshire, SP4 OJG, UK) on 22/9/00 under Provisional Accession No. 92209, 92210, 92211, and 92212, respectively. The antibodies produced by these hybridomas are further defined by the DNA sequence which encodes their light and heavy chains as recited in SEQ ID NO:281-288. The 4BE12C10 antibody can be obtained from the National Institute of Biological Standards and Control, Blanche Lane, South Mimms, Potters Bar, Herts, EN6 3QG, UK. The antibodies may be humanised or CDR grafted for therapeutic use using the sequences of SEQ ID NO:281-288 and techniques known in the art [see, for example, Holliger and Bohlen (1999) Cancer Metastasis Rev. 18:411-9, Gavilondo and Larrick (2000) Biotechniques 29:128-32, 134-6, 138, and Kipriyanov and Little (1999) Mol. Biotechnol. 12:173-201]. The term “antibody” herein is used to refer to a molecule having a useful antigen binding specificity. Those skilled in the art will readily appreciate that this term may also cover polypeptides which are fragments of the monoclonal antibodies of the invention which can show the same or a closely similar functionality. Such antibody fragments or derivatives are intended to be encompassed by the term antibody as used herein.

Mimotopes of L3,7,9 LOS that are capable of binding to the monoclonal antibodies of the invention, and immunogens comprising these mimotopes, form an important aspect of the present invention. Vaccines comprising mimotopes that are capable of binding to these antibodies are useful in the treatment or prevention of meningococcal disease.

Also forming an important aspect of the present invention is the use of the monoclonal antibodies of the invention in the identification of novel mimotopes of meningococcal L3,7,9 LOS, for subsequent use as an immunogen.

The present invention provides the use of novel peptides encompassing the epitopes or mimotopes of the present invention (as defined above), in the manufacture of pharmaceutical compositions for the prophylaxis or therapy of meningococcal disease. Immunogens comprising the mimotope or peptide of the present invention and a carrier molecule are also provided for use in vaccines for the immunoprophylaxis or therapy of meningococcal disease. Accordingly, the mimotopes, peptides or immunogens of the present invention are provided for use in a medicament, and in the medical treatment or prophylaxis of meningococcal disease. Accordingly, there is provided a method of treatment of meningococcal disease comprising the administration to a patient suffering from or susceptible to said disease, of a vaccine or medicament of the present invention.

Vaccines of the present invention may also include suitable excipients or diluents. Advantageously, an adjuvant is also included. Suitable adjuvants for vaccines of the present invention comprise those adjuvants that are capable of enhancing the antibody responses against the immunogen. Adjuvants are well known in the art (Vaccine Design—The Subunit and Adjuvant Approach, 1995, Pharmaceutical Biotechnology, Volume 6, Eds. Powell, M. F., and Newman, M. J., Plenum Press, New York and London, ISBN 0-306-44867-X). Preferred adjuvants for use with immunogens of the present invention include aluminium or calcium salts (for example hydroxide or phosphate salts). Other adjuvants include saponin adjuvants such as QS21 (U.S. Pat. No. 5,057,540) and 3D-MPL (GB 2220 211).

The vaccines of the present invention will be generally administered for both priming and boosting doses. It is expected that the boosting doses will be adequately spaced, or preferably given yearly or at such times where the levels of circulating antibody fall below a desired level. Boosting doses may consist of the peptide in the absence of the original carrier molecule. Such booster constructs may comprise an alternative carrier or may be in the absence of any carrier.

The vaccine preparation of the present invention may be used to protect or treat a mammal susceptible to, or suffering from meningococcal disease, by means of administering said vaccine via systemic or mucosal route. These administrations may include injection via the intramuscular, intraperitoneal, intradermal, transdermal or subcutaneous routes; or via mucosal administration to the oral/alimentary, respiratory, genitourinary tracts.

The amount of protein, peptide(s) or conjugated peptide(s) in each vaccine dose is selected as an amount which induces an immunoprotective response without significant, adverse side effects in typical vaccinees. Such amount will vary depending upon which specific immunogen is employed and how it is presented. Generally, it is expected that each dose will comprise 1-1000 μg of protein/peptide, preferably 1-500 μg, preferably 1-100 μg, of which 1 to 50 μg is the most preferable range. An optimal amount for a particular vaccine can be ascertained by standard studies involving observation of appropriate immune responses in subjects. Following an initial vaccination, subjects may receive one or several booster immunisations adequately spaced.

Aspects of the present invention may also be used in diagnostic assays. For example, the peptides or mimotopes of the present invention could be used to detect antibodies against L3,7,9 in the serum of a patient. Likewise the monoclonal antibodies of the invention could be used for detecting the presence of L3,7,9 immunotype meningococcus in a sample from a patient.

Vaccine preparation is generally described in New Trends and Developments in Vaccines, edited by Voller et al., University Park Press, Baltimore, Ma., U.S.A. 1978. Conjugation of proteins to macromolecules is disclosed by Likhite, U.S. Pat. No. 4,372,945 and by Armor et al., U.S. Pat. No. 4, 474,757.

An independent aspect of the invention is a vaccine against serogroup B, C, Y, or W-135 meningococci, which comprises a mimotope of a surface L3,7,9 LOS of N. meningitidis and a mimotope of a surface L2 LOS of N. meningitidis. Optionally, this vaccine may advantageously also comprise one or more plain or conjugated meningococcal capsular polysaccharides selected from a group comprising: C, Y and W-135.

A further aspect is a vaccine against serogroup A meningococci, which comprises a mimotope of a surface L3,7,9 LOS of N. meningitidis and a mimotope of a surface L10 LOS of N. meningitidis. Optionally, this vaccine may advantageously also comprise plain or conjugated N. meningitidis serogroup A capsular polysaccharide.

A further aspect still is a vaccine against serogroup A, B, C, Y, or W-135 meningococci, which comprises a mimotope of a surface L3,7,9 LOS of N. meningitidis, a mimotope of a surface L10 LOS of N. meningitidis and a mimotope of a surface L2 LOS of N. meningitidis. Optionally, this vaccine may advantageously also comprise one or more plain or conjugated meningococcal capsular polysaccharides selected from a group comprising: A, C, Y and W-135.

The inventors have found that the above formulations prove extremely effective in the immunoprotection of a mammalian host against the majority of strain variants encompassed within the above mentioned groups of meningococcal serotypes. Preferably, the mimotopes of the invention can be used to immunise a host such that antibodies are produced which specifically cross-react with LOS, and preferably cross-react with whole cell bacteria containing the LOS.

Each mimotope may be either peptidic or non-peptidic. Non-peptidic mimotopes are envisaged to be of a similar size, in terms of molecular volume, to their peptidic counterparts. Preferably, the mimotopes are antigenically cross-reactive with a monoclonal antibody of high specificity and/or affinity to the respective surface LOS. Preferably one or both mimotopes in the above vaccine combinations comprise a peptide epitope.

The peptide epitopes may be obtainable by screening a peptide library with a monoclonal antibody specific (and/or of high affinity) to the respective surface LOS. For the purposes of this invention, ‘high affinity’ typically means having an affinity constant of at least 10⁵M⁻¹, preferably a least 10⁶M⁻¹.

Monoclonal antibodies of high specificity and/or affinity to LOS may be prepared using outer membrane complexes as immunogens and detecting antigens according to established protocols (see for example Zollinger et al. 1983. I&I 40:257-264; Adbillahi et al. 1988. Microbial Pathogenesis 4:27-32).

The mimotope preferably comprises a peptide epitope which may be identified by screening a peptide library with the monoclonal antibody. Typically, a peptide library such as a heptapeptide or a nonapeptide (see above) library preferably containing all possible amino acid sequences should be used to give the greatest diversity of potential epitopes against which antigenic cross-reactivity with the monoclonal antibody can be assessed. Typically, a random peptide library of this nature is used.

Preferably the mimotopes are obtainable using cross-reactivity with the following monoclonal antibodies as a selection means: H44/24, H44/58, H44/70, H44/78, 4BE12C10, 4A8-B2 or 9-2-L397 for L3,7,9 LOS mimotopes; F1-5H 5/ID9 for L2 LOS mimotopes; and 5B4-F9-B10 for L10 LOS mimotopes.

H44/24, H44/58, H44/70, H44/78, and 4BE12C10 antibodies are described above. 4A8-B2, 9-2-L397, F1-5H 5/ID9 and 5B4-F9-B10 monoclonal antibodies may be obtained from the National Institute of Biological Standards and Control, Blanche Lane, South Mimms, Potters Bar, Herts, EN6 3QG, UK and may also be obtained through ECACC.

The peptide mimotopes of the above formulations may be conformationally constrained as described above. The mimotopes of each respective formulation may be contained within a single molecule. They may be linked to the same or different carrier molecules as described above. For instance they may be inserted within or substitute the same or different exposed loop region(s) of the same outer membrane protein of meningococcus (as described above).

The L3,7,9 mimotopes used in the formulations are preferably the minmotopes and peptides of the invention described above. Alternatively the mimotope of the L3,7,9 LOS can comprises a peptide disclosed in WO 00/25814, preferably selected from: IHRQGIH; HIGQRHI; LPARTEG; GETRAPL; APARQLP; PLQRAPA; KQAPVHH; HHVPAQK; LQAPVHH; HHVPAQL; LPSIQLP; PLQISPL; NELPHKL; LKHPLEN; KSPSMTL; LTMSPSK; AGPLMLL; LLMLPGA; WSPILLD DLLIPSW; LSMHPQN; NQPHMSL; HSMHPQN NQPHMSH; SMYGSYN; NYSGYMS; TNHSLYH; HYLSHNT; HAIYPRH; HRPYIAH; TTYSRFP; PFRSYTT; TDSLRLL; LLRLSDT; SFATNIP; and PINTAFS.

A preferred embodiment of the invention is a global vaccine which is particularly beneficial in the treatment or prevention of meningococcal disease comprising a mimotope of a surface L3,7,9 LOS of N. meningitidis, a mimotope of a surface L10 LOS of N. meningitidis, and a mimotope of a surface L2 LOS of N. meningitidis; optionally also comprising one or more plain or conjugated meningococcal capsular polysaccharides selected from a group comprising: A, C, Y and W-135.

A further preferred embodiment of the invention is a global vaccine which is particularly beneficial in the treatment or prevention of meningitis comprising the vaccine combinations described above (preferably that containing L3,7,9, L2 and L10 peptide mimotopes, and optionally one or more meningococcal capsular polysaccharides), and one or more plain or conjugated pneumococcal capsular polysaccharide antigens. The pneumococcal capsular polysaccharide antigens are preferably selected from serotypes 1, 2, 3, 4, 5, 6B, 7F, 8, 9N, 9V, 10A, 11A, 12F, 14, 15B, 17F, 18C, 19A, 19F, 20, 22F, 23F and 33F (most preferably from serotypes 1, 3, 4, 5, 6B, 7F, 9V, 14, 18C, 19F and 23F).

Yet another preferred combination of the invention is a global vaccine which is particularly beneficial in the treatment or prevention of meningitis comprising one or more (2 or 3) peptide mimotopes from the list consisting of L3,7,9, L2 and L10, and a conjugated H. influenzae b capsular polysaccharide.

The above vaccine combinations are suitable for use as a medicament, and may be used in the manufacture of a medicament for the treatment or prevention of meningococcal disease. A method for treating a patient suffering from or susceptible to meningococcal disease, comprising the administration of the above vaccine combinations to the patient is a further aspect of the invention.

### EXAMPLES

The examples below were carried out using standard techniques, which are well known and routine to those of skill in the art, except where otherwise described in detail. The examples are illustrative, but do not limit the invention.

### Example 1

### Monoclonal Antibodies Directed Against N. meningitidis L3,7,9 LOS

1.1 Isolation and Functional Characterization of the Monoclonal Antibodies

Immunization

Neisseria meningitidis B cells (heat inactivated cells from the H44/76 isolate, B:15:P1.7, 16, Los 3,7,9) were injected three times in BALB/C mice on days 0, 21 and 42 (5 animals/group). Cells, formulated in an oil-in-water/3D-MPL/QS21 adjuvant (as described in WO 95/17210), were injected both by the subcutaneous and intraperitoneal routes. Animals were evaluated 7 days after the third injection for antibody response in a whole cell Elisa.

Fusion Procedure to Obtain Specific Hybridoma Cell Lines

On days −10 and −7 before fusion, Sp2/0 Ag 14 myeloma cells were thawed and cultivated in a flask in order to reach and maintain a 10⁵ cells/ml culture up to the fusion day (day 0). On day −1, myeloma cell concentration was adjusted to 10⁵ cells/ml. At the same time, plates of feeder cells using peritoneal macrophages were prepared (10⁵ macrophages/ml; 100 μl/well).

On day 0, the spleen of the selected mouse was taken (under sterile conditions) and perfused with 10 to 20 ml of DMEM medium inside a Petri dish. Cells were counted (using a Sysmex counter and 2 drops of “Quicklyser” to lyse red cells) and centrifuged for 10 min. at 150-200 g (1000 rpm using the Beckman GPR centrifuge). Sp2/0 and spleen cells were washed, counted and centrifuged for 10 min at 150-200 g before mixing in a 1:5 ratio (Sp2/0 : spleen cells). The 1:5 mixing ratio was done in 25 ml DMEM and centrifuges for 5 min at 400 g (1500 rpm using the GPR centrifuge, 50-ml Falcon tube). The supernatant was discarded and the tube slightly tapped to put the pellet in suspension. The cells were kept as much as possible at 37° C., in a water-bath. One ml of PEG solution (PEG 4000 at 40% v/v with 5% de DMSO at pH 8.0-8.2), kept at 37° C., was slightly added (drop by drop) within 1 minute, while the tube was slightly shaken. The temperature of the cells had to be as close as possible to 37° C. From this PEG step, cells were manipulated gently. After 30 sec to 1 min, 1 ml of DMEM was added within 1 min, then 2 ml of DMEM within 2 min., and 4 ml DMEM within 4 min. Finally, the tube was filled with DMEM+additives in order to reach a volume of about 20 ml., and was centrifuged for 10 min. at 400 g.

Afterwards, the pellet was suspended gently in 15 to 25 ml of complete medium (DMEM, FCS+HS (Volker), HAT and Nutridoma) with a 25 ml pipet in order to break the aggregates.

Incubation of the. tube was done for 2 h. at 37° C. in a CO₂ incubator. The cells were then diluted to an adequate concentration (2.5 10⁴ to 10⁵ cells/well) and 100 μl of cells were plated in 96 wells microplates previously inoculated with feeder cells.

1.2 Recognition of Cell Surface Epitopes by Whole Cell ELISA

The homologous H44/76 MenB strain (B:15:P1.7, 16) was used as coated bacteria to detect specific anti-Neisseria meningitidis antibodies in animal sera, as well as in supernatants of hybridoma cultures after splenocyte fusion. Briefly, microtiter plates (Maxisorp, Nunc) were coated with 100 μl of a 1/10 dilution (in PBS) with a H44/76 bacteria solution from a 6 hours culture, in which bacteria were killed by 400 μg/ml tetracycline. Plates were incubated at 37° C. for at least 16 hours until plates were completely dried. Then, they were washed three times with 300 μl of 150 mM NaCl—0.05% Tween 20. Afterwards, plates were overcoated with 100 μl of PBS—0.3% casein and incubated for 30 min at room temperature with shaking. Plates were washed again using the same procedure before incubation with antibodies. Animal sera were serially two-fold diluted in PBS—0.3% Casein 0.05% Tween 20 and put into the microplates (12 dilutions starting at the 1/100 dilution) before incubation at room temperature for 30 min with shaking, before the next identical washing step. For screening of the monoclonal antibodies, supernatants were put as such (non diluted) in the microplates. Anti-mouse immunoglobulins (rabbit Ig, Dakopatts E0413) conjugated to biotin is used at 1/2000 in PBS—0.3% casein—0.05% Tween 20 to detect specific antibodies against several antigens at the cell surface. After the last washing step (as before), plates were incubated with a streptavidin-peroxidase complex solution diluted at 1/4000 in the same solution for 30 min at room temperature under shaking conditions. For characterization of the mabs, a few other Neisseria meningitidis B strains were also used as coated bacteria using the same procedure as described above: strains M97250 687 (B:4:P1.15) and M97252078 isolated in UK from human beings. Transformed H44/76D cells lacking the capsular polysaccharide and having a mutated LOS were also used by whole cell Elisa to characterize these mabs (see the next paragraph). Reactivity of the antibodies in whole cell ELISA ranged from no detected signal (−) to strong reactivity (+++). ps 1.3 Strain Transformation

The plasmid pMF121 (Frosch et al., 1990) was used to construct a Neisseria meningitidis serogroup B strain lacking the capsular polysaccharide. This plasmid contains the erythromycin resistance gene flanked by recombination regions corresponding to the ends of gene cluster encoding the group B polysaccharide (B PS) biosynthetic pathway. Deletion of the B PS resulted in loss of expression of the group B capsular polysaccharide as well as a deletion in the active copy of galE leading to the synthesis of galactose deficient lipo-oligosaccharide (LOS).

Neisseria meningitidis serogroupe B strain H44/76 (B:15:P1.7, 16; LOS 3,7,9) was used for transformation. After an overnight CO₂ incubation on Muller-Hinton (MH) plate (without erythromycin), cells were collected in liquid MH containing 10 mM MgCl₂ (2 ml were used per MH plate) and diluted up to an OD of 0.1 (550 nm). To this 2 ml solution, 4 μl of the plasmid pMF121 stock solution (0.5 μg/ml) were added for a 6 hours incubation period at 37° C. (with shaking). A control group was done with the same amount of Neisseria meningitidis bacteria, but without addition of plasmid. After the incubation period, 100 μl of culture, as such, at 1/10, 1/100 and 1/1000 dilutions, were put in MH plates containing 5, 10, 20, 40 or 80 μg erythromycin/ml before incubation for 48 hours at 37° C.

Colony Blotting:

After plate incubation, transformants were selected and grown onto erythromycin/ MH plates (10 to 80 μg erythromycin/ml). The day after, all the visible colonies were placed on new MH plates without erythromycin in order to let them grow. Then, they were transferred onto nitrocellulose sheets (colony blotting) and probed for the presence of B polysaccharide. Briefly, colonies were plotted onto a nitrocellulose sheet and rinsed directly in PBS—0.05% Tween 20 before cell inactivation for 1 hour at 56° C. in PBS—0.05% Tween 20 (diluant buffer). Afterwards, the membrane was overlaid for one hour in the diluant buffer at room temperature (RT). Then, membranes were washed again for three times 5 minutes in the diluant buffer before incubation with the anti-B PS 735 Mab (From Dr Frosch, via Boerhinger) diluted at 1/3000 in the diluant buffer for 2 hours at RT. After a new washing step (3 times 5 minutes), the monoclonal antibody was detected with a biotinylated anti-mouse Ig from Amersham (RPN 1001) diluted 500 times in the diluant buffer (one hour at RT) before the next washing step (as described above). Afterwards, sheets were incubated for one hour at RT with a solution of streptavidin-peroxidase complex diluted 1/1000 in the diluant buffer. After the last three washing steps using the same method, nitrocellulose sheets were incubated for 15 min in the dark using the revelation solution (30 mg of 4-chloro-1-naphtol solution in 10 ml methanol plus 40 ml PBS and 30 mcl of H₂O₂ 37% from Merck). The reaction was stopped with a distilled water-washing step. Clones lacking reactivity with the anti-B PS Mab were further characterized by whole cell ELISA.

Whole Cell ELISA:

Whole cell ELISAs were also done using the transformed colonies and the wild type strain (H44/76) as coated bacteria (20 μg protein/ml), and a set of different monoclonal antibodies used to characterize Neisseria meningitidis strains. The following Mabs were tested: anti-B PS (735 from Dr Frosch), and the other Mabs from the National Institute for Biological Standards and Control, London: anti-B PS (Ref 95/750) anti-P1.7 (A-PorA, Ref 4025), anti-P1.16 (A-PorA, Ref 95/720), anti-Los 3,7,9 (A-LPS, Ref 4047), anti-Los 8 (A-LPS, Ref 4048), and anti-P1.2 (A-PorA Ref 95/696).

Microtiter plates (Maxisorp, Nunc) were coated with 100 μl of the recombinant meningococcal B cells solution overnight (ON) at 37° C. at around 20 μg/ml in PBS. Afterwards, plates are washed three times with 300 μl of 150 mM NaCl—0.05% Tween 20, and were overlaid with 100 μof PBS—0.3% Casein and incubated for 30 min at room temperature with shaking. Plates were washed again using the same procedure before incubation with antibodies. Monoclonal antibodies (100 μl) were used at different dilutions in PBS—0.3% Casein 0.05% Tween 20 and put onto the microplates before incubation at room temperature for 30 min with shaking, before the next identical washing step. 100 μl of the anti-mouse Ig (from rabbit, Dakopatts E0413) conjugated to biotin and diluted at 1/2000 in PBS—0.3% Casein—0.05% Tween 20 were added to the wells to detect bound monoclonal antibodies. After the washing step (as before), plates were incubated with a streptavidin-peroxidase complex solution (100 μl of the Amersham RPN 1051) diluted at 1/4000 in the same working solution for 30 min at room temperature under shaking conditions. After this incubation and the last washing step, plates are incubated with 100 μl of the chromogenic solution (4 mg orthophenylenediamine (OPD) in 10 ml 0.1 M citrate buffer pH4.5 with 5 μl H₂O₂) for 15 min in the dark. Plates are then read at 490/620 nm using a spectrophotometer.

Routinely, about 10% of the transformants resulted from double cross-over. Bacterial clone H44/76D lacking reactivity against BPS & LOS but still reactive against P1.7 &P1.16 mAbs was selected for further studies.

1.4 Bactericidal Assay

The bactericidal activity of these monoclonal antibodies was measured. Briefly, the Neisseria meningitidis serogroup B (H44/76 strain as a first strain) is used to determine the bactericidal activity of the antibodies (animal or human sera or monoclonal antibodies). In U-bottom 96 well microplates (NUNC), 50 μl/well of serial two-fold serum dilutions were incubated with 37.5 μl/well of the log phase meningococcal suspension adjusted to 2.5 10⁴ CFU/ml and incubated for 15 min at 37° C. with shaking at 210 rpm (Orbital shaker, Forma Scientific). Then, 12.5 μl of the baby rabbit complement (Pel-freeze Biologicals, US) is added before incubation for one more hour in the same conditions. Afterwards, 10 μl aliquots of the mixture from each well were spot onto Mueller-Hinton agar plates containing 1% Isovitalex and 1% of heat inactivated Horse serum before overnight incubation at 37° C. with 5% CO₂. The day after, colonies are counted for each dilution tested and bactericidal titers determined as the dilution of the serum for 50% killing, compared with the complement control without serum. By this method, individual colonies can be counted up to 100 CFU per spot. Titers are expressed as the dilution which induce 50% killing, calculated by regression analysis.

1.5 Results

Table 1 illustrates results obtained with 4 anti-LOS monoclonal antibodies from two fusion experiments with splenocytes from mice immunized with Neisseria meningitidis strain H44/76. The LOS specificity of the 4 monoclonal antibodies is supported by their failure to react with the H44/76D (BPS and LOS mutated) strain in whole cell ELISA, whereas these readily reacted with the wild type H44/76 strain. Considering the non-immunogenic nature of the BPS, it is extremely unlikely that these monoclonal antibodies react with the capsular polysaccharide. All 4 monoclonal antibodies exhibited bactericidal activity against the wild type strain H44/76. Monoclonal antibodies H44/24 and H44/58 were shown to cross-react with M97250687 and M97252078 N. meningitidis strains by whole cell ELISA.

TABLE 1

| | | Bactericidal | Recognition of | | | |
|---|---|---|---|---|---|---|
| | | activity | N. meningitidis strains by whole cell ELISA | | | |
| Anti- | Iso- | against strain | H44/76 (wild | H44/76D | | |
| body | type | H44/76 (titer) | type) | (mutant) | M97250687 | M97252078 |
| H44/24 | IgG3 | >2560 | +++ | − | +++ | +++ |
| H44/58 | IgG1 | 973 | +++ | − | +++ | ++ |
| H44/70 | IgM | 1884 | +++ | − | NT | NT |
| H44/78 | IgM | >2560 | +++ | − | NT | NT |

NT. = not tested

1.6 Structural Characterization of the 4 Selected Monoclonal Antibodies

The primary structure of the monoclonal antibodies was determined by sequencing the cDNA encoding the variable heavy and light chains. To achieve this, total RNA was extracted from the hybridomas producing those 4 monoclonal antibodies. Sequencing has been carried out on RT-PCR products obtained following PCR using primers specific for heavy or light chains.

Extraction of RNA from Hybridomas

Extraction is performed on 10⁶ cells as determined after counting in a Thoma cell. Cells are centrifuged 10 minutes at 1200RPM and supernatant is removed. Cells are centrifuged again 2 minutes at 1200RPM and all traces of supernatant are removed. Cells are resuspended in 200 μl RNAse-free PBS. RNA extraction is performed with the “High Pure RNA Isolation Kit” (Roche Diagnostics) according to the manufacturers instructions. Elution is performed in 100 μl elution buffer.

Reverse Transcription

10 μl of the purified RNA were mixed with 1.25 μg dT15 primer in a 20 μl final volume. The RNA-primer mix was heated for 10 minutes at 70° C. and then cooled to 4° C. The reverse transcription was realized using the following protocol:

In a 0.2 ml tube were added:

10 μl of the above annealed primer-RNA mix

5 μl of 0.1M DTT

2.5 μl of dNTPs (10 mM each)

0.5 μl RNase inhibitor (10 u/μl, GibcoBRL)

2.5 μl M-MLV Reverse Transcriptase (200U/μl, GibcoBRL)

10μl RT buffer (5×)

H₂O to 50μl

This was incubated for 1 hour at 37° C., the enzyme was inactivated for 5 minutes at 95° C. and cooled on ice. A tube containing the same components but no M-MLV Reverse Transcriptase was used as a negative control.

PCR on the cDNA

The primers used for the PCR amplification of the light and heavy chains cDNAs were designed according to Kang et al. (Kang, et al. 1991 Methods. A companion to Methods in Enzymology 2(2): 111-118):

| | | | |
|---|---|---|---|
| Light chain | 3′ primer | L-Kappa3′ | 5′ GCGCCGTCTAGAATTAACACTCATTCCTGTTGAA 3′ |
| | 5′ primers | Lvar 5′-1 | 5′ CCAGTTCCGAGCTCGTTGTGACTCAGGAATCT 3′ |
| | | Lvar 5′-2 | 5′ CCAGTTCCGAGCTCGTGTGACGCAGCCGCCC 3′ |
| | | Lvar 5′-3 | 5′ CCAGTTCCGAGCTCGTGCTCACCCAGTCTCCA 3′ |
| | | Lvar 5′-4&7 | 5′ CCAGTTCCGAGCTC(G/C)(A/T)GATGAC(A/C)CAGTCTCCA 3′ |
| | | Lvar 5′-5&6 | 5′ CCAGATGTGAGCTCGT(G/C)ATGACCCAG(A/T)CTCCA 3′ |
| Heavy chain | 5′ primers | Hvar 5′ 1-8 | 5′ AGGTCCA(A/G)CT(G/T)CTCGAGTC(A/T)GG 3′ |
| | | Hvar 5′ 9 | 5′ AGGTIIAICTICTCGAGTC(A/T)GG 3′ |
| | 3′ primers | HCγ 3 3′ | 5′ GGGGGGTACTAGTCTTGGGTATTCTAGGCTC 3′ |
| | | HCμ 3′ | 5′ ATTGGGACTAGTTTCTGCGACAGCTGGAAT 3′ |

PCR was accomplished as follows:

Mix in a 0.2 ml reaction tube:

5 μl template (RT-PCR product or negative control)

5 μl reaction buffer (10×)

1 μl dNTPs (10 mM each)

1.5 μl each primer (20 μM stocks)

2.5 μl REDTaq polymerase (1U/μl, Sigma)

H₂O to 50 μl

The PCR reaction was performed with the following temperature cycling:

4 min at 94° C.

35 times 30 seconds at 94° C.

30 seconds at 52° C.

1 min at 72° C.

4 min at72° C.

4° C.

These reactions were also performed on the negative controls from RT reactions (without M-MLV Reverse Transcriptase) to be sure that the PCR products are obtained from cDNA and not genomic DNA. No PCR products were obtained from those negative controls. The obtained PCR products were purified with the “High Pure PCR Product Purification Kit” (Roche Diagnostics) according to the manufacturer's instructions.

Sequencing of the Purified PCR Products

To be sure that no sequence errors occurred because the sequencing was performed on a PCR product, sequencing was done on two independently-obtained PCR products for each cDNA to be sequenced.

Sequencing reactions were performed on 1 μl of the purifed PCR products with the “ABI PRISM® BigDye™ Terminator Cycle Sequencing Kit” according to the manufacturer's instructions (Perkin-Elmer) and analyzed on a ABI PRISM 377 sequencer. Sequences of the light and heavy chains for the 4 monoclonal antibodies are presented in SEQ ID NO:281-288.

### Example 2

### Isolation of N. meningitidis LOS Peptidic Mimotopes from Phage-displayed Peptidic Libraries

Monoclonal antibodies directed against epitopes on bacterial polysaccarides, as are the above-mentioned antibodies directed against N. meningitidis LOS, can be used to screen large repertoires of molecules. Such molecular libraries can chemically different, for example, peptides, peptoids, or nucleotides. Peptidic libraries can be obtained either synthetically (as soluble or support-bound peptides) or biologically (for example as fusions to a cytoplasmic or surface protein). One of the most often used systems is the display of peptides fused to a coat protein of filamentous bacteriophages such as the pIII and pVIII proteins. These libraries are obtained by inserting an oligonucleotide containing a degenerate sequence in the 5′ region of the ORF encoding one of these 2 proteins. The peptides expressed at the surface of the phage in fusion with the pIII or pVIII proteins are physically linked to their encoding DNA since the filamentous phages consist of the phage circular single-stranded DNA surrounded by the structural proteins. Two phage-displayed peptidic libraries were used in this work for selection of peptides with five monoclonal antibodies. These two libraries are the nonamer linear and nonamer disulfide-constrained peptide libraries previously described (Felici et al. 1993 Gene 128: 21-27; Luzzago et al. 1993 Gene 128: 51-57). These libraries are constructed in a phagemid, the degenerate oligonucleotides being fused to the gene encoding the major capsid protein, pvIII. After transformation in E. coli and superinfection with phage helper, the resulting phage particles contain both recombinant and non-recombinant pVIII proteins, the proportion of recombinant pVIII proteins not being precisely defined. The five monoclonal antibodies used for selection of peptides are the four described above, and the monoclonal antibody 4BE12C10 obtained from Ian Feavers (National Institute for Biological Standards and Control, London).

4BE12C10 was also used to select peptides from a mix of 4 other libraries. These libraries express peptides 14 to 16 amino acids in length fused to the pVIII protein of the f88-4 filamentous phage. This vector, received from Goerges Smith (Division of Biological Sciences, University Of Missouri-Columbia), has two gene VIIIS: the wild-type gene and a synthetic recombinant gene under the control of the tac promoter, and is derived from the fd-tet phage (Smith, Virology, 167, 156-165, 1988). After infection of E. coli, the resulting phage particles contain both wild-type and recombinant pVIII proteins, the recombinant pVIII protein being a few to 10% of all pVIII proteins. The 4 libraries express peptides that contain two internal cysteines separated by 3, 4, 5 or 6 residues and are called Cys3, Cys4, Cys5 and Cys6, respectively.

2.1 Panning Procedures

Three cycles of panning were performed. For mAbs H44/24 and H44/58, two procedures were used for immobilisation of phage-antibodies complexes: capture on ProteinA-coated immunoplate (hereunder refered as procedure PA) or capture on ProteinA-coated magnetic beads (hereunder refered as procedure DY). For mAbs H44/70, H44/78 and 4BE12C10, phage-antibodies complexes were recovered by capture on ProteinLA-coated immunoplates.

2.1.1 ProteinA-coated Immunoplates (PA) Procedure (for H44/24 and H44/58)

During the first cycle, Maxisorp immunoplates (Nunc, Roskilde, Denmark) were coated overnight at 4° C. with ProteinA (Sigma, St. Louis, USA) at 10 μg/ml of 0.1 M sodium carbonate (four wells with 100 μl ProteinA solution for each mAb to be used). At the same time, a sample of the libraries mix (5.10¹⁰ pfu for each of the two libraries) was incubated overnight at 4° C. with 10 μg of mAb in the smallest possible volume (typically less than 40 μl). The following day, after 1 hr saturation (5mg/ml BSA, 0.1 μg/ml ProteinA in 0.1M sodium carbonate) and 4 washes with Tris Buffered Saline (TBS, 50 mM Tris, 150 mM NaCl, pH 7.5) containing Tween20 0.5% (v/v), the antibody-phages mixes were filled up to 400 μl with the washing solution and four 100 μl aliquotes were incubated for 3 hours at room temperature on the proteinA-coated dishes. After 10 washes with TBS-Tween20 0.5%, bound phages were eluted for 20 minutes at room temperature with a glycine buffer at pH 2.2. The eluate was immediately neutralized and used for amplification and titration of infectious phage particles. The E. coli strain used for amplification and titration was DH11S (GibcoBRL). For the following cycles of biopanning, the same protocol was used but the amount of mAb was reduced to 1 μg.

2.1.2 ProteinA-coated Magnetic Beads (DY) Procedure (for H44/24 and H44/58)

For the first cycle, a sample of the libraries mix (5.10¹⁰ pfu for each of the two libraries) was incubated overnight at 4° C. with 10 μg of mAb in the smallest possible volume (typically less than 40 μl). The following day, 40 μl Dynabeads ProteinA (Dynal, Oslo, Norway) were washed 2 times with TBS-Tween, saturated for 1 hr (5 mg/ml BSA, 0.1 μg/ml ProteinA in 0,1M sodium carbonate) and washed 2 more times with TBS-Tween. The antibody-phage mixes were filled up to 40 μl with the washing solution, mixed with the saturated Dynabeads ProteinA and incubated 3 hours at room temperature. After 10 washes with TBS-Tween20 0.5%, bound phages were eluted 20 minutes at room temperature with a glycine buffer at pH 2.2. The eluate was treated as in Example 2.1.1. For the following cycles of biopanning, the same protocol was used but the amount of mAb was reduced to 1 μg.

2.1.3 ProteinLA-coated Immunoplates Procedure (for H44/70, H44/78 and 4BE12C10)

The protocol used for this procedure is the same that for the ProteinA-coated immunoplate procedure except that ProteinA was replaced by ProteinLA (Clontech, Palo Alto, USA) and that non-purified concentrated hybridoma supernatant was used.

2.1.4 Selection from the Cys3 to Cys6 Library Mix with Monoclonal Antibody 4BE12C10.

The protocol was similar to the PA procedure discussed above in 2.1.1, except the following:

3.10¹⁰ TU of each library were used in the first panning monoclonal antibody 4BE12C10 was used in three different amounts for the three panning cycles: 10 μg for the first cycle, 1 μg for the second cycle and 0.1 μg for the third cycle.

ProteinG (10 μg/ml) was used to capture the phage-antibody complexes in the first and third panning rounds, and ProteinA (10 μg/ml) was used to capture the phage-antibody complexes in the second panning round.

2.2 Amplification of Phages

Phage eluates from the nonamer libraries were amplified by infection of E. coli (DH11S) and superinfection with helper phage M13KO7. A sample of the eluates (450 μl out of the total 475 μl of the first panning round or 100 μl out of the total 475 μl of the second or third panning rounds) was mixed with 1 ml of terrific broth cells (DH11 S grown in terrific broth at OD600 nm between 0.125 and 0.25 at dilution 10). Bacteria were kept 15 minutes at 37° C. with slow agitation just before and after infection. Infected bacteria were then grown 30 minutes at 37° C. with strong agitation and then spread on large LB plates supplemented with 100 μg/ml ampicillin (LB Amp). The next day, the plates were scraped and a sample was added to 100 ml LB Amp to reach 0.05 OD600 nm. This culture was grown to a OD600 nm between 0.2 and 0.25, the agitation was slowed down for 10 minutes and superinfection by helper phage was performed by adding M13KO7 at a MOI (multiplicity of infection) of 20. At this time, IPTG was added at a final concentration of 1 mM. The culture was incubated 15 minutes at 37° C. without agitation and grown 5 hours at 37° C. with strong agitation. Phages were recovered by precipitation of cleared supernatant with PEG-NaCl and titrated by infection of E. coli (DH11S) and spreading on LB Amp plates.

Phage eluates from the Cys3/4/5/6 libraries were amplified by infection of E. coli (K91kan). A sample of the eluates (450 μl out of the total 475 μl of the first panning round or 100 μl out of the total 475 μl of the second or third panning rounds) was mixed with 1 ml of terrific broth cells (K91kan grown in terrific broth at OD600 nm between 0.125 and 0.25 at one tenth dilution). Bacteria were kept 15 minutes at 37° C. with slow agitation just before and after infection. Infected bacteria were then grown 30 minutes at 37° C. with strong agitation in LB medium supplemented with 0.2 μl g/ml tetracycline. Tetracycline was then added up to 20 μg/ml and grow overnight at 37° C. with strong agitation. Phages were recovered by precipitation of cleared supernatant with PEG-NaCl and titrated by infection of E. coli (K91kan) and spreading on LB Tet plates.

2.3 Screening by Immunoblottings

2.3.1 Screening by Colony Immunoblotting

Screening by colony immunoblotting was done using E. coli (DH11S) infected with phage obtained after 3 panning cycles. Seven phage samples were used: one sample of phage selected with each of the monoclonal antibodies H44/70, H44/78 and 4 BE12C10, and two samples for each of the monoclonal antibodies H44/24 and H44/58. In the latter case, the two samples correspond to the two methods used for the pannings (refered as procedures PA and DY above).

Petri dishes containing ampicillin (100 μg/ml) and 1 mM IPTG were used for spreading the phage-infected E. coli onto. Fresh colonies were blotted with nylon amphoteric membranes (Porablot NY amp, Macherey-Nagel, Düren, Germany) for 2 hours at 37° C. The membranes were subsequently saturated with 5% skimmed milk in TBS (2 hours at 37° C.) and incubated with the corresponding monoclonal antibody. The binding of the monoclonal antibody to the recombinant pVIII proteins was revealed using GAM-HRP (Dako, Denmark) diluted 1000 times in saturation solution. The presence of the secondary antibody was in turn detected using HRP color development reagent (Bio-Rad, Hercules, USA).

2.3.2 Screening by Culture Supernatant Immunoblotting

In the case of the screening of the two nonamer libraries with monoclonal antibody 4BE12C10, no positive clone could be revealed by the colony immunoblotting technique from either the second or third round of panning. In order to try to isolate some individual positive clones, a new experiment was set up which consisted of isolating 48 clones from each panning round and producing phages from these isolated clones in duplicate in a 96-well format allowing a quick isolation of positive clones in a culture supernatant immunoblot experiment. Culture and phage production by superinfection with M13KO7 helper phage were carried out as described in point 2.2 except that culture volumes were reduced to 500 μl to afford the 96-well format. Hundred microlitre of the culture supernatants were directly blotted on a nitrocellulose membrane in a 96-well format dot blotting device (BioRad) and treated as membranes of the colony immunoblotting experiment.

Monoclonal antibody binding was revealed by a chemiluminescent detection technique: membrane was incubated 10 minutes with LumiLight Plus substrate (Roche Diagnostics) at room temp. in the dark, and light emission was detected by putting the membrane in contact with an autoradiography film for 30 seconds to a few minutes in an autoradiography cassette. A similar blot was incubated with an anti-phage serum to check for the presence of phage particles in similar amounts in all phage-containing wells.

2.4 Sequence Determination of Selected Peptides

The sequence of the selected peptides was determined using a two-step procedure. In the first step, the recombinant gene 8 was amplified by polymerase chain reaction (PCR) using oligonucleotides annealing upstream (5′-ATTCTAGAGATTACGCC-3′ for the nonamer libraries or 5′-CCCATCCCCCTGTTGACAAT-3′ for the Cys3/4/5/6 libraries) and downstream (5′-TGCTGCAAGGCGATTAAGTT-3′ for the nonamer libraries or 5′-ATTAGGCGGGCTGGGTATCT-3′ for the Cys3/4/5/6 libraries) of the region coding for the presented peptide. The PCR were carried out with the Tth thermostable DNA polymerase (BIOTOOLS, Madrid, Spain) in duplicate in order to check for possible risks or errors during amplification. The PCR products were sequenced using with the M13-40 Forward primer (5′-GTTTTCCCAGTCACGAC-3′ ) (for peptides derived from the nonamer libraries) or with 5′-CATCGGCTCGTATAATGT-3′ (for peptides derived from the Cys3/4/5/6 libraries) using the “ABI PRISM® BigDye™ Terminator Cycle Sequencing Kit” according to the manufacturer's instructions (Perkin-Elmer) and analyzed on a ABI PRISM 377 sequencer.

148 different sequences were obtained distributed as follows:

1 peptide selected with monoclonal antibodies H44/24, H44/58, H44/70 and H44/78 (sequence N^{o} 1)

11 peptides selected only with monoclonal antibody H44/24 (either by procedure DY or procedure PA) (sequences N^{o} 2 to 12)

30 peptides selected only with monoclonal antibody H44/58 (either by procedure DY or procedure PA) (sequences N^{o} 13 to 43, except 17)

1 peptide selected with monoclonal antibody H44/58 (either by procedure DY or procedure PA) and monoclonal antibody H44/70 (sequence N^{o} 17)

3 peptides selected with monoclonal antibodies H44/70 and H44/78 (sequences N^{o} 44 to 46)

28 peptides selected only with monoclonal antibody H44/70 (sequences N^{o} 47 to 74)

21 peptides selected only with monoclonal antibody H44/78 (sequences N^{o} 75 to 95)

53 peptides selected only with monoclonal antibody 4 BE12C10 (sequences N^{o} 96 to 148). Sequences 141 and 142 are from phage derived from the cysteine-bridged nonamer library. Sequences 143 to 145 are from phage derived from the linear nonamer library. Sequences 146 to 148 are from phage derived from the Cys6, Cys5 and Cys3 libraries, respectively.

These sequences are presented in Table 2 below.

TABLE 2
Sequences of the 148 selected phage-displayed
peptides.

| No. | Sequence | SEQ ID |
|---|---|---|
| 1 | CNTKYPYAC | 141 |
| 2 | CFVPSPYVYEC | 142 |
| 3 | CRSSLPGDC | 143 |
| 4 | FYRELAGDL | 4 |
| 5 | MRRTASEIM | 5 |
| 6 | MRPLTWQTT | 6 |
| 7 | RMRIIPEGT | 7 |
| 8 | MRDVMPQHW | 8 |
| 9 | HKPTDHPSW | 9 |
| 10 | CSETYGRPGLC | 150 |
| 11 | ERPIGGDSG | 11 |
| 12 | RMRDIPGAP | 12 |
| 13 | CISEYAKGTTC | 153 |
| 14 | CSHAPPYDRVC | 154 |
| 15 | CVTIPYRGTQC | 155 |
| 16 | CFAPPYDPLPC | 156 |
| 17 | CAPYSIFIGEC | 157 |
| 18 | CTHLYHYGTSC | 158 |
| 19 | CLCQAYKGRRC | 159 |
| 20 | CDPRLLDLC | 160 |
| 21 | KTALPPYDR | 21 |
| 22 | CFARPFQGTWC | 162 |
| 23 | CSLSLPPYDRC | 163 |
| 24 | CDRTLSALALC | 164 |
| 25 | CRAPPYDTIMC | 165 |
| 26 | CPPYDEGCRVA | 26 |
| 27 | CFGLIAFHPDC | 167 |
| 28 | CQPIGPPYDRC | 168 |
| 29 | CTANVYFGTYC | 169 |
| 30 | CANSRPGGYLC | 170 |
| 31 | CMSSYGRGVRC | 171 |
| 32 | CVSTPFRGTFC | 172 |
| 33 | CDPRITPDFGC | 173 |
| 34 | CGPPYDPFPAC | 174 |
| 35 | CHTVRFRGTLC | 175 |
| 36 | CTAPPYDAYGC | 176 |
| 37 | CRSPLLGAPVC | 177 |
| 38 | CTTTYGTGTWC | 178 |
| 39 | CSSLYYHGTAC | 179 |
| 40 | CLYEPLRGTLC | 180 |
| 41 | CAPPPYDQSFC | 181 |
| 42 | CPPPWYSRSSC | 182 |
| 43 | CSRALGYVSEC | 183 |
| 44 | PTWYKLKSV | 44 |
| 45 | RGSEGSFAR | 45 |
| 46 | CKQTIGSFDGC | 186 |
| 47 | PLWYDPAPP | 47 |
| 48 | ESPYSAHRW | 48 |
| 49 | WYDERTILK | 49 |
| 50 | CSSYSYVHDSC | 190 |
| 51 | CRFTYDPPFMC | 191 |
| 52 | CRLYSFVFDKC | 192 |
| 53 | SQWRSAAPT | 53 |
| 54 | CRPAFDPPYHC | 194 |
| 55 | HGRTLWYTP | 55 |
| 56 | CSSVSATYPIC | 196 |
| 57 | CSLVQSPKRFC | 197 |
| 58 | SNWYENTPT | 58 |
| 59 | PRPGWGQSA | 59 |
| 60 | CTDPRGCGMFA | 60 |
| 61 | PRPHFGAPP | 61 |
| 62 | CVTRATYPSWC | 202 |
| 63 | WYIAPRKTL | 63 |
| 64 | CYGYSALRDTC | 204 |
| 65 | CIITGSGWYVC | 205 |
| 66 | CTHYSFYGDIC | 206 |
| 67 | HWYSTEAAW | 67 |
| 68 | HRIAQSLPQ | 68 |
| 69 | CALYRFAADSC | 209 |
| 70 | CRPQFDPPNDC | 210 |
| 71 | CHPALARWPLC | 211 |
| 72 | QPKSLWYSV | 72 |
| 73 | CRGYSHVSDAC | 213 |
| 74 | CDPVRTIYPIC | 214 |
| 75 | WYTTPTRPV | 75 |
| 76 | QRQSLWYSS | 76 |
| 77 | NPDYSSPHE | 77 |
| 78 | PPWYPEHKT | 78 |
| 79 | CASLGLAKTTC | 219 |
| 80 | WYVDGPLAT | 80 |
| 81 | RGWYADPSA | 81 |
| 82 | CLWRPIDPFLC | 222 |
| 83 | AERSLWYYP | 83 |
| 84 | PPWYNQSEL | 84 |
| 85 | DSAPAVKSS | 85 |
| 86 | PGWYDAHPT | 86 |
| 87 | CRGLQGHIAYC | 227 |
| 88 | WYSAPENAL | 88 |
| 89 | WYTAPSLSL | 89 |
| 90 | WYTNPSIAA | 90 |
| 91 | WYFSNENLG | 91 |
| 92 | WYTLDIGPT | 92 |
| 93 | GPWHGPSSS | 93 |
| 94 | GDWPPFSAP | 94 |
| 95 | WYVGSVRSQ | 95 |
| 96 | CALDIAGGYIC | 236 |
| 97 | CPPPSRGGYIC | 237 |
| 98 | CQAFDTSWTAC | 238 |
| 99 | FLPCRRCGS | 99 |
| 100 | RPWQTAHFA | 100 |
| 101 | GQYSSSPFP | 101 |
| 102 | CGRPGPYPADC | 242 |
| 103 | CTPLPDGGILC | 243 |
| 104 | LKWGDGSSA | 104 |
| 105 | CYPQLSHANPC | 245 |
| 106 | CSAYHRSLGAC | 246 |
| 107 | CFPLPSREFAC | 247 |
| 108 | YRQSRSSWP | 108 |
| 109 | SHRFDALRR | 109 |
| 110 | CVRFPDGSHSC | 250 |
| 111 | CSPAAFSDRLC | 251 |
| 112 | CVTDQWGGYLC | 252 |
| 113 | CVPSGRSPNTC | 253 |
| 114 | PKWSDKRPQ | 114 |
| 115 | CAPPGIAVRTC | 255 |
| 116 | MKWGPNSHS | 116 |
| 117 | CLQDRAGGYLC | 257 |
| 118 | CGRLEGRCSHA | 118 |
| 119 | CYFIAKHGWAC | 259 |
| 120 | CPPRSSRGFLC | 260 |
| 121 | CTGISTGEYLC | 261 |
| 122 | GPVFYATGL | 122 |
| 123 | CLSQYADWTYC | 263 |
| 124 | ARWYPISQT | 124 |
| 125 | CQGFPGAPQDC | 265 |
| 126 | WHFRTFPAT | 126 |
| 127 | TRRPFDPPA | 127 |
| 128 | CASPLGPCFW | 128 |
| 129 | CWTDTYGDLLC | 269 |
| 130 | CISAGPESSHC | 270 |
| 131 | CHSVQPATRAC | 271 |
| 132 | CPKAPFSPFKC | 272 |
| 133 | CIDAGSHGWLC | 273 |
| 134 | CRRGSPLSRYC | 274 |
| 135 | SWDEIIDLG | 135 |
| 136 | CRSPAGEWSSC | 276 |
| 137 | CAYHVLRYSAC | 277 |
| 138 | CAKTVRGDYYC | 278 |
| 139 | CLAASADTAAC | 279 |
| 140 | CPYTSWAREGC | 280 |
| 141 | CPPPWSTHDC | 297 |
| 142 | CSEPWSTSNC | 298 |
| 143 | AITGVRARW | 291 |
| 144 | EKKHFNYGT | 292 |
| 145 | EKKRFESNT | 293 |
| 146 | EQGYCTVNIEQCAKYR | 294 |
| 147 | SPADCDYTTLCAKPT | 295 |
| 148 | NSPTSCKWLCNEKF | 296 |

### Example 3

### ELISA Tests for the Peptide-on-phage mAb Interaction

To assess the binding of the mAbs to the numerous peptides-on-phage, all clones have been produced independently using the following protocol.

For Clones from the Nonamer Libraries (Table 2 Peptide Nos 1 to 145).

A preculture of E. coli DH11S infected with the phage was grown overnight. A sample of this preculture was used to inoculate a new 50 ml culture with a starting OD₆₀₀ₙₘ of 0.050. This culture was grown up to an OD₆₀₀ₙₘ of 0.20 to 0.25 with vigorous shaking at 37° C. The culture was then slowed down for 15 minutes in order to allow the regeneration of pili. M13K07 helper phage was added at an MOI of 20, and superinfection was allowed for 15 more minutes at 37° C. with slow agitation. IPTG (1 mM final concentration) was then added and the culture was grown for 5 hours with vigorous agitation.

For Clones from the Cys3/4/5/6 Libraries (Table 2, Peptide Nos. 146 to 148).

A colony of K91kan containing the clone of interest (on a LB Tet plate) was added to 50 ml of LB supplemented with 20 μg/ml tetracycline and grown overnight at 37° C. with strong agitation.

For all Clones.

Culture was centrifuged for 15 minutes at 4000RPM; 0.15 volume of PEG-NaCl solution was added to the supernatant for precipitating the phage and kept overnight at 4° C. Phage were collected by centrifugation, 45 minutes at 4000RPM. After being resuspended in TBS, phage were heated 15 minutes at 65° C. to kill remaining bacteria and to denature soluble proteins present in the sample. The solution was then centrifuged and the pellet was discarded. Phage in the supernatant were then precipitated again as indicated above. Phage were finally resuspended in 200 μl of TBS. Concentration of phage particules was determined by measuring the ΔOD at 269 nm-320 nm.

The ELISA Test

Phages were coated at 5×10¹¹ particules/ml on MaxiSorp multiwell plates. Coating was performed overnight at 4° C. with 100 μl/well. Plates were saturated with 5% (v/v) skimmed milk in TBS for 2 hours at 37° C. and washed 5 times with TBS-Tween₂₀ 0.05% (v/v). The mAbs were then incubated in the coated wells for 1 hour at 37° C., washed 5 times, and GAM-HRP conjugate (1500-fold dilution, Dako, Denmark) was added for 1 hour at 37° C. to detect the binding of the mAb to the phages. After 5 washings, the peroxidase activity was monitored by addition of the K-Blue® substrate (Neogen, Lexington, USA) at room temperature for 20 minutes. Reaction was stopped by addition of 25 μl of 2N H₂SO₄. Reading was performed at 450-630 nm. Each phage was coated in triplicate for testing with each mAb. Control mAbs are of the same isotype as the selecting mAbs but do not react with N. meningitidis B.

Results

All phages that were positive with one or more than one mAb were tested a second time to confirm the result. Forty-six peptides were shown to be positive with at least one mAb. These are peptide number (with reference to Table 2): 13, 14, 17, 22, 27, 28, 29, 30, 39, 45, 47, 50, 51, 53, 54, 55, 58, 61, 63, 66, 75, 82, 83, 85, 86, 87, 88, 93, 103, 104, 105, 115, 124, 131, 132, 133, 139, 140, 141, 142, 143, 144, 145, 146, 147, 148. This supports the view that they are particularly suitable as mimotopes of the meningococcal L3,7,9 LOS.

Hundred and two out of the 148 selected peptides were not detected as positive in this test. For at least some of these, this could be due to bad expression of the peptide at the surface of the phage, especially for those peptides which share a conserved motif with other peptides that are positive in the test. In such case, a more sensitive test may show that some of these peptides are indeed recognized by one or more mAbs.

### Example 4

### SPOT Peptides

Another experiment for determining the best peptide candidates for immunisation trials is whether chemically synthesized peptides are recognized by at least one anti-MenB LOS mAb (and preferably not by irrelevant mAbs). This can be assessed on SPOT synthesized peptides (peptides synthesized directly on a cellulose membrane). This membrane can be tested with different mAbs by repetitive immunoblottings and chemiluminescent detection. Peptides may be synthesised with 3 residues originating from the pVIII protein sequence on each end of the peptide. Indeed, the primary structure of the peptides expressed in fusion with pVIII on the surface of the bacteriophage is as follows (x for any residue in the library):

linear peptides (9aa): AAEGEFxxxxxxxxxDPAKAAF . . .

cyclic peptides (C9aaC): AAEGEFCxxxxxxxxxCGDPAKAAF . . .

Linear and cyclic peptides may be synthesised on distinct membranes to enable specific regeneration of cyclic peptides.

For example linear peptides comprising peptide No. 61 from Table 2 (GEFPRPHFGAPPDPA) and peptide No. 83 (GEFAERSLWYYPDPA) were synthesised on one membrane, and cyclised peptides comprising peptide No. 50 from Table 2 (GEFCSSYSYVHDSCGDP), peptide 14 (GEFCSHAPPYDRVCGDP) and peptide 25 (GEFCRAPPYDTIMCGDP) were synthesised on another.

The protocol used to probe these membranes with mAbs was the one provided by Genosys, with some modifications, especially for the regeneration of membranes with cyclised peptides. Briefly, membranes were washed 3×10 minutes (washing buffer is TTBS: TBS pH8, Tween 20, 0,05%) saturated for 1 hour at room temp. in blocking buffer (Blocking buffer concentrate Genosys SU-07-250, 10 times diluted in TTBS, 0,05g/ml sucrose), washed again for 1 minute in TTBS, incubated for 2 hours (rocking gently) at room temp. with mAb diluted in blocking buffer, washed 2×1 minute and 3×10 minutes with TTBS, incubated 1h30 (rocking gently) at room temp. with secondary antibody (Goat anti-mouse-HRP, Dako) diluted 1500× in blocking buffer, washed 2×1 minute and 3×10 minutes with TTBS, and measured by chemiluminescent detection (incubated 10 minutes with LumiLight Plus (Roche Diagnostics) at room temp. in the dark and light emission was detected on a Fluor-S MultiImager System (BioRad) for 3 to 30 seconds or by autoradiography on X-ray film).

Results are reported in the tables below. First column is the number of the peptide. MAbs (in bold), are indicated in the order they were used in the successive experiments.

Mabs used: MAb 24 (H44/24), 58 (H44/58), 47 (4BE12C10) and 2C8 (an irrelevant mAb) are IgG3; MAbs 70 (H44/70), 78 (H44/78), M4 (directed against streptococcal polysaccharide), M13 (id.) and F76 (generated against a peptide epitope) are IgMs. G stands for GAM-HRP alone (no primary mAb).

Membrane 1 (linear peptide)

| | 70 | Gam | 78 | 47 | M4 | 58 | M13 | 24 | G | 2C8 | F76 |
|---|---|---|---|---|---|---|---|---|---|---|---|
| 83 | +++ | | + | (+) | ++ | (+) | ++ | ++ | | | ++ |
| 61 | + | | | | | | | | | | +/− |

Membrane 2 (cyclic peptides)

| | 58 | G | 24 | 70 | 78 | M13 | 47 | 70 | 2C8 | F76 |
|---|---|---|---|---|---|---|---|---|---|---|
| 14 | ++ | | | + | | + | | | | (+) |
| 25 | (+) | | | | | (+) | | | | (+) |
| 50 | | | | ++ | + | + | | ++ | | (+) |

After detection, blots were regenerated as follows: washed 3×10 minutes in TTBS; washed 2×1 minute and 3×10 minutes in regeneration buffer at 50° C. (regeneration buffer is: 50 mM Tris-HCl, pH 6.7; 2% SDS; 2-mercaptoethanol 100 mM); washed 3×10 minutes in PBS (only for cyclic peptides); washed 2×1 minutes and then overnight in PBS-DMSO 10% (only for cyclic peptides); and washed 3×10 minutes in TTBS.

From the experiment it can be concluded that Peptide 61 is specifically recognized by mAb H44/70 and Peptide 50 is recognized by mAb H44/70, H44/78 and M13 but the signal level is higher with mAb H44/70 than with other mAbs. Peptide 83 is recognized by all IgMs but the signal is higher with mAb H44/70 than with other mAbs. This peptide is also recognized by mabs H44/24 and H44/58 and not by other IgG3. Peptides 14 and 25, positive only with mAb H44/58 in phage-ELISA, were positive in SPOT synthesis with the same mAb and no other IgG3.

### Example 5

### Analysis of the Peptide Structure

The peptides isolated in this study appear to be quite dissimilar to the set of L3,7,9 LOS peptide mimotopes disclosed in WO 00/25814.

148 peptides were found in this study (see Table 2). Many of them have, by design, one Cys residue at both ends, to make them cyclic via a disulphide bridge. In order to avoid, in the alignment and pattern search procedures, any bias due to these Cys, the peptides have been trimmed of their terminal cysteines; the original presence of cysteines in the peptides can be recognized in the last two characters of their names (CC stands for one Cys at both ends, CN stands for one cys at N-ter, and no cys at C-ter, NC is the reverse, and NN when no terminal cys at any end).

The average amino acid composition of the peptides has two striking features: they seem to be enriched in Prolines (98/148 have at least one Proline) and even more so in aromatic residues (Tyr, Trp and Phe) (126 peptides have at least one aromatic residue).

In particular, seven peptides (1, 2, 18, 50, 64, 83, 123) have the motif [YW]xY, reported to be able to mimic carbohydrate subunits (C. D. Partidos, Current Opinion in Mol. Therapeutics, Vol 2, pp74-79, 2000).

One might think that the presence of a disulfide bridge would favour prolines in the peptides, but the unconstrained peptides contain prolines almost as frequently.

Conserved patterns were searched for, with the following results.

Strong Similaritys Shared by Some Peptides

Some peptides share a number of consecutive residues, as shown in the following table:

| Number of | | Peptide |
|---|---|---|
| peptides | Motif shared | numbers |
| 2 | L-P-P-Y-D-R | 21, 23 |
| 4 | P-P-Y-D-R | 14, 21, 23, 28 |
| 4 | A-P-P-Y-D | 14, 16, 25, 36 |
| 2 | P-P-Y-D-P | 16, 34 |
| 2 | G-P-P-Y-D | 28, 34 |
| 2 | A-G-G-Y-L | 96, 117 |
| 2 | S-L-W-Y-S | 72, 76 |
| 2 | S-R-S-S | 42, 108 |
| 3 | P-P-W-Y | 42, 78, 84 |
| 10 | P-P-Y-D | See below |
| 2 | R-G-T-L | 35, 40 |
| 3 | F-D-P-P | 54, 70, 127 |
| 2 | A-T-Y-P | 56, 62 |
| 2 | P-G-A-P | 12, 125 |
| 2 | G-R-P-G | 10, 102 |
| 2 | F-R-G-T | 32, 35 |
| 4 | G-G-Y-L | 30, 96, 112, 117 |
| 2 | S-L-S-L | 23, 89 |
| 3 | S-L-W-Y | 72, 76, 83 |

One should particularly notice the large number (10) of peptides sharing the PPYD motif:

| | |
|---|---|
| LTpep_14_CC | shaPPYDrv |
| LTpep_16_CC | faPPYDplp |
| LTpep_21_NN | ktalPPYDr |
| LTpep_23_CC | slslPPYDr |
| LTpep_25_CC | raPPYDtim |
| LTpep_26_CN | PPYDegcrv |
| LTpep_28_CC | qpigPPYDr |
| LTpep_34_CC | gPPYDpfpa |
| LTpep_36_CC | taPPYDayg |
| LTpep_41_CC | apPPYDqsf |

There are 17 peptides having the motif PP[YFW].

The doublet WY is especially frequent in the set, with a count of 26 peptides. The next most frequent dipeptide is PP (24) followed by AP (20).

Weaker Similarities Shared by Many Peptides

If intervening mismatches are accepted, the following sets of related peptides are found:

| | |
|---|---|
| LTpep_54_CC | RPaFDPPyh |
| LTpep_70_CC | RPqFDPPnd |
| LTpep_96_CC | alDiAGGYL |
| LTpep_117_CC | lqDrAGGYL |
| LTpep_89_NN | WYTaPSlsl |
| LTpep_90_NN | WYTnPSiaa |
| LTpep_78_NN | PpWYpeHkT |
| LTpep_86_NN | PgWYdaHpT |
| LTpep_7_NN | RMRiIPegt |
| LTpep_12_NN | RMRdIPgap |
| LTpep_16_CC | faPPYDPlP |
| LTpep_34_CC | gPPYDPfPa |
| LTpep_52_CC | RlYSfVfDk |
| LTpep_73_CC | RgYShVsDa |
| LTpep_47_NN | PlWYdpapp |
| LTpep_86_NN | PgWYdahPt |
| LTpep_78_NN | PpWYpehkt |
| LTpep_86_NN | PgWYdahpt |
| LTpep_42_CC | pPpWYsrss |
| LTpep_84_NN | PpWYnqsel |
| LTpep_75_NN | WYTtPtrpv |
| LTpep_89_NN | WYTaPslsl |
| LTpep_90_NN | WYTnPsiaa |
| LTpep_63_NN | WYiAPrktL |
| LTpep_88_NN | WYsAPenaL |
| LTpep_89_NN | WYtAPslsL |

Allowing for weaker patterns, we can group the last two sets with the pattern WYxxP, and add another peptide:

| | |
|---|---|
| LTpep_81_NN | rgWYadpsa |
| LTpep_63_NN | WYiaPrkal |
| LTpep_88_NN | WYsaPenal |
| LTpep_75_NN | WYttPtrpv |
| LTpep_89_NN | WYtaPslsl |
| LTpep_90_NN | WYtnPsiaa |

If some more flexibility is introduced by allowing for gaps in the alignments, even more peptides may be grouped; for example:

| | |
|---|---|
| LTpep_54_CC | RPaFDPPyh |
| LTpep_70_CC | RPqFDPPnd |
| LTpep_127_NN | trRP-FDPPa |
| LTpep_21_NN | ktAl-PPYDr |
| LTpep_41_CC | Ap-PPYDqsf |
| LTpep_54_CC | rpAfdPPYH |
| LTpep_47_NN | plWYd-Papp |
| LTpep_55_NN | hgrtlWYt-P |
| LTpep_63_NN | WYiaPrktl |
| LTpep_75_NN | WYttPtrpv |
| LTpep_81_NN | rgwYadPsa |
| LTpep_83_NN | aerslwYy-P |
| LTpep_88_NN | WYsaPenal |
| LTpep_89_NN | WYtaPslsl |
| LTpep_90_NN | WYtnPsiaa |

Other consensus sequences:

| | |
|---|---|
| Peptide 141 | cppPWSThdc |
| Peptide 142 | csePWSTsnc |
| Consensus | PWST |
| Peptide 144 | EKKhFnygT |
| Peptide 145 | EKKrFesnT |
| Consensus | EKKxFxxxT |

A Comparison of the Selected Peptides Selected in Example 3

Two thirds of the peptides are constrained by a disulphide bridge, which means that this feature is not necessarily essential.

| | |
|---|---|
| NTKWYPYA | SEQ ID NO:1 |
| FVPSPYVYE | SEQ ID NO:2 |
| RSSLPGD | SEQ ID NO:3 |
| FYRELAGDL | SEQ ID NO:4 |
| MRRTASEIM | SEQ ID NO:5 |
| MRPLTWQTT | SEQ ID NO:6 |
| RMRIIPEGT | SEQ ID NO:7 |
| MRDVMPQHW | SEQ ID NO:8 |
| HKPTDHPSW | SEQ ID NO:9 |
| SETYGRPGL | SEQ ID NO:10 |
| ERPIGGDSG | SEQ ID NO:11 |
| RMRDLPGAP | SEQ ID NO:12 |
| ISEYAKGIT | SEQ ID NO:13 |
| SHAFPYDRV | SEQ ID NO:14 |
| VTIPYRGTQ | SEQ ID NO:15 |
| FAPPYDPLP | SEQ ID NO:16 |
| APYSIFIGE | SEQ ID NO:17 |
| THLYHYGTS | SEQ ID NO:18 |
| LCQAYKGRR | SEQ ID NO:19 |
| DPRLLDL | SEQ ID NO:20 |
| KTALPPYDR | SEQ ID NO:21 |
| FARPFQGTW | SEQ ID NO:22 |
| SLSLPPYDR | SEQ ID NO:23 |
| DRTLSALAL | SEQ ID NO:24 |
| RAPPYDTIM | SEQ ID NO:25 |
| CPPYDEGCRVA | SEQ ID NO:26 |
| FGLLAFHPD | SEQ ID NO:27 |
| QPIGPPYDR | SEQ ID NO:28 |
| TANYYFGTY | SEQ ID NO:29 |
| ANSRPGGYL | SEQ ID NO:30 |
| MSSYGRGVR | SEQ ID NO:31 |
| VSTPFRGTF | SEQ ID NO:32 |
| DPRITPDFG | SEQ ID NO:33 |
| GPPYDPFPA | SEQ ID NO:34 |
| HTVRFRGTL | SEQ ID NO:35 |
| TAPPYDAYG | SEQ ID NO:36 |
| RSPLLGAPV | SEQ ID NO:37 |
| TTTYGTGTW | SEQ ID NO:38 |
| SSLYYHGTA | SEQ ID NO:39 |
| LYEPLRGTL | SEQ ID NO:40 |
| APPPYDQSF | SEQ ID NO:41 |
| PPPWYSRSS | SEQ ID NO:42 |
| SRALGYVSE | SEQ ID NO:43 |
| PTWYKLKSV | SEQ ID NO:44 |
| RGSEGSFAR | SEQ ID NO:45 |
| KQTIGSFDG | SEQ ID NO:46 |
| PLWYDPAPP | SEQ ID NO:47 |
| ESPYSAHRW | SEQ ID NO:48 |
| WYDERTILK | SEQ ID NO:49 |
| SSYSYVHDS | SEQ ID NO:50 |
| RFTYDPPFM | SEQ ID NO:51 |
| RLYSFVFDK | SEQ ID NO:52 |
| SQWRSAAPT | SEQ ID NO:53 |
| RPAFDPPYH | SEQ ID NO:54 |
| HGRTLWYTP | SEQ ID NO:55 |
| SSVSATYPI | SEQ ID NO:56 |
| SLVQSPKRF | SEQ ID NO:57 |
| SNWYENTPT | SEQ ID NO:58 |
| PRPGWGQSA | SEQ ID NO:59 |
| CTDPRGCGMFA | SEQ ID NO:60 |
| PRPHFGAPP | SEQ ID NO:61 |
| VTRATYPSW | SEQ ID NO:62 |
| WYIAPRKTL | SEQ ID NO:63 |
| YGYSALRDT | SEQ ID NO:64 |
| IITGSGWYV | SEQ ID NO:65 |
| THYSFYGDI | SEQ ID NO:66 |
| HWYSTEAAW | SEQ ID NO:67 |
| HRIAQSLPQ | SEQ ID NO:68 |
| ALYRFAADS | SEQ ID NO:69 |
| RPQFDPPND | SEQ ID NO:70 |
| HPALARWPL | SEQ ID NO:71 |
| QPKSLWYSV | SEQ ID NO:72 |
| RGYSHVSDA | SEQ ID NO:73 |
| DPVRTIYPI | SEQ ID NO:74 |
| WYTTPTRPV | SEQ ID NO:75 |
| QRQSLWYSS | SEQ ID NO:76 |
| NPDYSSPHE | SEQ ID NO:77 |
| PPWYPEHKT | SEQ ID NO:78 |
| ASLGLAKTT | SEQ ID NO:79 |
| WYVDGPLAT | SEQ ID NO:80 |
| RGWYADPSA | SEQ ID NO:81 |
| LWRPIDPFL | SEQ ID NO:82 |
| AERSLWYYP | SEQ ID NO:83 |
| PPWYNQSEL | SEQ ID NO:84 |
| DSAPAVKSS | SEQ ID NO:85 |
| PGWYDAHPT | SEQ ID NO:86 |
| RGLQGHIAY | SEQ ID NO:87 |
| WYSAPENAL | SEQ ID NO:88 |
| WYTAPSLSL | SEQ ID NO:89 |
| WYTNPSIAA | SEQ ID NO:90 |
| WYFSNEnLG | SEQ ID NO:91 |
| WYTLDIGPT | SEQ ID NO:92 |
| GPWHGPSSS | SEQ ID NO:93 |
| GDWPPFSAP | SEQ ID NO:94 |
| WYVGSVRSQ | SEQ ID NO:95 |
| ALDIAGGYI | SEQ ID NO:96 |
| PPPSRGGYI | SEQ ID NO:97 |
| QAFDTSWTA | SEQ ID NO:98 |
| FLPCRRCGS | SEQ ID NO:99 |
| RPWQTAHFA | SEQ ID NO:100 |
| GQYSSSPFP | SEQ ID NO:101 |
| GRPGPYPAD | SEQ ID NO:102 |
| TPLPDGGIL | SEQ ID NO:103 |
| LKWGDGSSA | SEQ ID NO:104 |
| YPQLSHANP | SEQ ID NO:105 |
| SAYHRSLGA | SEQ ID NO:106 |
| FPLPSREFA | SEQ ID NO:107 |
| YRQSRSSWP | SEQ ID NO:108 |
| SHRFDALRR | SEQ ID NO:109 |
| VRFPDGSHS | SEQ ID NO:110 |
| SPAAFSDRL | SEQ ID NO:111 |
| VTDQWGGYL | SEQ ID NO:112 |
| VPSGRSPNT | SEQ ID NO:113 |
| PKWSDKRPQ | SEQ ID NO:114 |
| APPGIAVRT | SEQ ID NO:115 |
| MKWGPNSHS | SEQ ID NO:116 |
| LQDRAGGYL | SEQ ID NO:117 |
| CGRLEGRCSHA | SEQ ID NO:118 |
| YFLAKHGWA | SEQ ID NO:119 |
| PPRSSRGFL | SEQ ID NO:120 |
| TGISTGEYL | SEQ ID NO:121 |
| GPVFYATGL | SEQ ID NO:122 |
| LSQYADWTY | SEQ ID NO:123 |
| ARWYPISQT | SEQ ID NO:124 |
| QGFPGAPQD | SEQ ID NO:125 |
| WHFRTFPAT | SEQ ID NO:126 |
| TRRPFDPPA | SEQ ID NO:127 |
| CASPLGPCFW | SEQ ID NO:128 |
| WTDTYGDLL | SEQ ID NO:129 |
| ISAGPESSH | SEQ ID NO:130 |
| HSVQPATRA | SEQ ID NO:131 |
| PKAPFSPFK | SEQ ID NO:132 |
| IDAGSHGWL | SEQ ID NO:133 |
| RRGSPLSRY | SEQ ID NO:134 |
| SWDEIIDLG | SEQ ID NO:135 |
| RSPAGEWSS | SEQ ID NO:136 |
| AYHVLRYSA | SEQ ID NO:137 |
| AKTVRGDYY | SEQ ID NO:138 |
| LAASADTAA | SEQ ID NO:139 |
| PYTSWAREG | SEQ ID NO:140 |
| CNTKWYPYAC | SEQ ID NO:141 |
| CFVPSPYVYEC | SEQ ID NO:142 |
| CRSSLPGDC | SEQ ID NO:143 |
| CFYRELAGDLC | SEQ ID NO:144 |
| CMRRTASEIMC | SEQ ID NO:145 |
| CMRPLTWQTTC | SEQ ID NO:146 |
| CRMRIIPEGTC | SEQ ID NO:147 |
| CMRDVMPQHWC | SEQ ID NO:148 |
| CHKFTDHPSWC | SEQ ID NO:149 |
| CSETYGRPGLC | SEQ ID NO:150 |
| CERPIGGDSGC | SEQ ID NO:151 |
| CRMRDIPGAPC | SEQ ID NO:152 |
| CISEYAKGTTC | SEQ ID NO:153 |
| CSHAPPYDRVC | SEQ ID NO:154 |
| CVTIPYRGTQC | SEQ ID NO:155 |
| CFAPPYDPLPC | SEQ ID NO:156 |
| CAPYSIFIGEC | SEQ ID NO:157 |
| CTHLYHYGTSC | SEQ ID NO:158 |
| CLCQAYKGRRC | SEQ ID NO:159 |
| CDPRLLDLC | SEQ ID NO:160 |
| CKTALPPYDRC | SEQ ID NO:161 |
| CFARPFQGTWC | SEQ ID NO:162 |
| CSLSLPPYDRC | SEQ ID NO:163 |
| CDRTLSALALC | SEQ ID NO:164 |
| CRAPPYDTIMC | SEQ ID NO:165 |
| CPPYDEGCRVAC | SEQ ID NO:166 |
| CFGLIAFHPDC | SEQ ID NO:167 |
| CQPIGPPYDRC | SEQ ID NO:168 |
| CTANYYFGTYC | SEQ ID NO:169 |
| CANSRPGGYLC | SEQ ID NO:170 |
| CMSSYGRGVRC | SEQ ID NO:171 |
| CVSTPFRGTFC | SEQ ID NO:172 |
| CDPRITPDFGC | SEQ ID NO:173 |
| CGPPYDPFPAC | SEQ ID NO:174 |
| CHTVRFRGTLC | SEQ ID NO:175 |
| CTAPPYDAYGC | SEQ ID NO:176 |
| CRSPLLGAPVC | SEQ ID NO:177 |
| CTTTYGTGTWC | SEQ ID NO:178 |
| CSSLYYHGTAC | SEQ ID NO:179 |
| CLYEPLRGTLC | SEQ ID NO:180 |
| CAPPPYDQSFC | SEQ ID NO:181 |
| CPPPWYSRSSC | SEQ ID NO:182 |
| CSRALGYVSEC | SEQ ID NO:183 |
| CPTWYKLKSVC | SEQ ID NO:184 |
| CRGSEGSFARC | SEQ ID NO:185 |
| CKQTIGSFDGC | SEQ ID NO:186 |
| CPLWYDPAPPC | SEQ ID NO:187 |
| CESPYSAHRWC | SEQ ID NO:188 |
| CWYDERTILKC | SEQ ID NO:189 |
| CSSYSYVHDSC | SEQ ID NO:190 |
| CRFTYDPPFMC | SEQ ID NO:191 |
| CRLYSFVFDKC | SEQ ID NO:192 |
| CSQWRSAAPTC | SEQ ID NO:193 |
| CRPAFDPPYHC | SEQ ID NO:194 |
| CHGRTLWYTPC | SEQ ID NO:195 |
| CSSVSATYPIC | SEQ ID NO:196 |
| CSLVQSPKRFC | SEQ ID NO:197 |
| CSNWYENTPTC | SEQ ID NO:198 |
| CPRPGWGQSAC | SEQ ID NO:199 |
| CTDPRGCGMFAC | SEQ ID NO:200 |
| CPRPHFGAPPC | SEQ ID NO:201 |
| CVTRATYPSWC | SEQ ID NO:202 |
| CWYIAPRKTLC | SEQ ID NO:203 |
| CYGYSALRDTC | SEQ ID NO:204 |
| CIITGSGWYVC | SEQ ID NO:2O5 |
| CTHYSFYGDIC | SEQ ID NO:206 |
| CHWYSTEAAWC | SEQ ID NO:207 |
| CHRIAQSLPQC | SEQ ID NO:208 |
| CALYRFAADSC | SEQ ID NO:209 |
| CRPQFDPPNDC | SEQ ID NO:210 |
| CHPALARWPLC | SEQ ID NO:211 |
| CQPKSLWYSVC | SEQ ID NO:212 |
| CRGYSHVSDAC | SEQ ID NO:213 |
| CDPVRTIYPIC | SEQ ID NO:214 |
| CWYTTPTRPVC | SEQ ID NO:215 |
| CQRQSLWYSSC | SEQ ID NO:216 |
| CNPDYSSPHEC | SEQ ID NO:217 |
| CPPWYPEHKTC | SEQ ID NO:218 |
| CASLGLAKTTC | SEQ ID NO:219 |
| CWYVDGPLATC | SEQ ID NO:220 |
| CRGWYADPSAC | SEQ ID NO:221 |
| CLWRPLDPFLC | SEQ ID NO:222 |
| CAERSLWYYPC | SEQ ID NO:223 |
| CPPWYNQSELC | SEQ ID NO:224 |
| CDSAPAVKSSC | SEQ ID NO:225 |
| CPGWYDAHPTC | SEQ ID NO:226 |
| CRGLQGHIAYC | SEQ ID NO:227 |
| CWYSAPENALC | SEQ ID NO:228 |
| CWYTAPSLSLC | SEQ ID NO:229 |
| CWYTNPSIAAC | SEQ ID NO:230 |
| CWYFSNEnLGC | SEQ ID NO:231 |
| CWYTLDIGPTC | SEQ ID NO:232 |
| CGPWHGPSSSC | SEQ ID NO:233 |
| CGDWPPFSAPC | SEQ ID NO:234 |
| CWYVGSVRSQC | SEQ ID NO:235 |
| CALDIAGGYIC | SEQ ID NO:236 |
| CPPPSRGGYIC | SEQ ID NO:237 |
| CQAFDTSWTAC | SEQ ID NO:238 |
| CFLPCRRCGSC | SEQ ID NO:239 |
| CRPWQTAHFAC | SEQ ID NO:240 |
| CGQYSSSPFPC | SEQ ID NO:241 |
| CGRPGPYPADC | SEQ ID NO:242 |
| CTPLPDGGILC | SEQ ID NO:243 |
| CLKWGDGSSAC | SEQ ID NO:244 |
| CYPQLSHANPC | SEQ ID NO:245 |
| CSAYHRSLGAC | SEQ ID NO:246 |
| CFPLPSREFAC | SEQ ID NO:247 |
| CYRQSRSSWPC | SEQ ID NO:248 |
| CSHRFDALRRC | SEQ ID NO:249 |
| CVRFPDGSHSC | SEQ ID NO:250 |
| CSPAAFSDRLC | SEQ ID NO:251 |
| CVTDQWGGYLC | SEQ ID NO:252 |
| CVPSGRSPNTC | SEQ ID NO:253 |
| CPKWSDKRPQC | SEQ ID NO:254 |
| CAPPGIAVRTC | SEQ ID NO:255 |
| CMKWGPNSHSC | SEQ ID NO:256 |
| CLQDRAGQYLC | SEQ ID NO:257 |
| CGRLEGRCSHAC | SEQ ID NO:25S |
| CYFIAKHGWAC | SEQ ID NO:259 |
| CPPRSSRGFLC | SEQ ID NO:260 |
| CTGISTGEYLC | SEQ ID NO:261 |
| CGPVFYATGLC | SEQ ID NO:262 |
| CLSQYADWTYC | SEQ ID NO:263 |
| CARWYPLSQTC | SEQ ID NO:264 |
| CQGFPGAPQDC | SEQ ID NO:265 |
| CWHFRTFPATC | SEQ ID NO:266 |
| CTRRPFDPPAC | SEQ ID NO:267 |
| CASPLGPCFWC | SEQ ID NO:268 |
| CWTDTYGDLLC | SEQ ID NO:269 |
| CISAGPESSHC | SEQ ID NO:270 |
| CHSVQPATRAC | SEQ ID NO:271 |
| CPKAPFSPFKC | SEQ ID NO:272 |
| CIDAGSHGWLC | SEQ ID NO:273 |
| CRRGSPLSRYC | SEQ ID NO:274 |
| CSWDEIIDLGC | SEQ ID NO:275 |
| CRSPAGEWSSC | SEQ ID NO:276 |
| CAYHVLRYSAC | SEQ ID NO:277 |
| CAKTVRGDYYC | SEQ ID NO:278 |
| CLAASADTAAC | SEQ ID NO:279 |
| CPYTSWAREGC | SEQ ID NO:280 |
| PPPWSTHD | SEQ ID NO:289 |
| SEPWSTSN | SEQ ID NO:290 |
| AITGVRARW | SEQ ID NO:291 |
| EKKHFNYGT | SEQ ID NO:292 |
| EKKRFESNT | SEQ ID NO:293 |
| EQGYCTVNIEQCAKYR | SEQ ID NO:294 |
| SPADCDYTTLCAKPT | SEQ ID NO:295 |
| NSPTSCKWLCNEKF | SEQ ID NO:296 |
| CPPPWSTHDC | SEQ ID NO:297 |
| CSEPWSTSNC | SEQ ID NO:298 |
| CAITGVRARWC | SEQ ID NO:299 |
| CEKKHFNYGTC | SEQ ID NO:300 |
| CEKKRFESNTC | SEQ ID NO:301 |

Sequence of the light chain of the H44/24 monoclonal antibody
SEQ ID NO:281
5′GACCCAGTTTCCACTTTTCCCTGCCTGTCAGTCTTGGAGATCAAACCTCCATCTCTTGCACATCTA
GTCGGAGCCTTGTACACCGGTAAAGGAAACACCTATTTACATTGGTACCTTCAGAAGCCAGGCCAGTC
TCCAAAGCTCCTGATCTACAAAGTTTCCAACCGATTTTCTGGGGTCCCAGACAGGTTCAGTGGCAGTG
GATCAGGGACAGATTTCACACTCACGATCAGCAGAGTGGAGGCTGAGGATCTGGGACTTTATTTCTGC
TCTCAAAGTACACATGTTCCGTGGACGTTCGGTGGAGGCACCAACCTGGAAATCAAACGGGCTGATGC
TGCACCAACTGTATCCATCTTCCCACCATCCAGTGAGCAGTTAACATCTGGAGGTGCCTCAGTCGTGT
GCTTCTTGAACAACTTCTACCCCAAAGACATCAATGTCAAGTGGAAGATTGATGGCAGTGAACGACAA
AATGGCGTCCTGAACAGTTGGACTGATCAGGACAGCAAAGACAGCACCTACAGCATGAGCAGCACCCT
CACGTTGACCAAGGACGAGTATGAACGACATAACAGCTATACCTGTGAGGCCACTCACAAGACATCAA
CTTCACCCATTGTCAAGAGCG 3′
Sequence of the heavy chain of the H44/24 monoclonal
antibody
SEQ ID NO:282
5′CCTGAGTCTGAAGGTGGCCTGGTGCACCcTGAAGgATCCCTGAAACTCTCCTGTGCAGCCTCCGGA
TTCGATTTTAGTAGATACTGGATGAGTTGGGTCCGGCAGGCTCCAGGGAAAGGGCTAGAATGGATTGG
AGAAATTTATCCAGATAGCAGAACGATAAACTATACGCCTTCTCTAAAGGATAAATTCATCATCTCCA
GAGACAACGCCAAAAATACGCTATACCTGCAAATGAGCAAAGTGAGATCTGAGGACACAGGCCTTTAT
TACTGTGCAATCTACTTTACTTACGACGCCTGGGTTATGGACTACTGGGGTCAAGGAACCTCAGTCAC
CGTCTCCTCAGCTACAACAACAGCCCCATCTGTCTATCCCTTGGTCCCTGGCTGCAGTGACACATCTG
GATCCTCGGTGACACTGGGATGCCTTGTCAAAGGCTACTTCCCTGAGCCGGTAACTGTAAAATGGAAC
TATGGAGCCCTGTCCAGCGGTGTGCGCACAGTCTCATCTGTCCTGCAGTCTGGGTTCTATTCCCTCAG
CAGCTTGGTGACTGTACCCTCCAGCACCTGGCCCAGCCAGACTGTCATCTGCAACGTAGCCCACCCAG
CCAGCAAGACTGAGTTgTCAAGA 3′
Sequence of the light chain of the H44/58 monoclonal antibody
SEQ ID NO:283
5′CCCAGTTCCACTtTCCTGCcTGTCAGTCTTGgAGATCAAGCcTCCATcTCTTGCAGATCTAGTCAG
AGCCTTGTTCACAATAAGGGAAACACCTATTTACATTGGTaCCTGCAGAAGCCAGGCCAGTcTCCAAA
GCTCCTGATCTACAAAGTTTCCAACCGATTTTCTGGGGTCCCTGACAGGTTCAGTGGCAGTGATTCAG
GGACAGATTTCACACTCAGGATCAGCAGAGTGGAGGCTGAGGATCTGGGAGTTTATTTCTGCTCTCAA
AGTACACATGTTCCGTGGACGTTCGGTGGAGGCACCAAGCTGGAAATCAAACGGGCTGATGCTGCACC
AACTGTATCCATCTTCCCACCATCCAGTGAGCAGTTAACATCTGGAGGTGCCTCAGTCGTGTGCTTCT
TGAACAACTTCTACCCCAAAGACATCAATGTCAAGTGGAAGATTGATGGCAGTGAACGACAAAATGGC
GTCCTGAACAGTTGGACTGATCAGGACAGCAAAGACAGCACCTACAGCATGAGCAGCACCCTCACGTT
GACCAAGGACGAGTATGAACGACATAACAGCTATACCTGTGAGGCCACTCACAAGACATCAACTTCAC
CCATTGTCAAGAGCG 3′
Sequence of the heavy chain of the H44/58 monoclonal
antibody
SEQ ID NO:284
5′TCTGAAGTGGCTGGTGCAGCTGAAGGATCCCTGAAaCTcTCCTGTGTCGCCACAGGATTCGATTTT
AGTAGATACTGGATGAGTTGGGTCCGGCAGGCTCCAGGGAAAGGGCTAGAGTGGATTGGAGAAATTTA
TCCAGATAGCAGGAAGATAAACTATACGCCATCTCTAAAGGATAAGTTCATCATCTCCAGAGACAACG
CCAAAAATACGCTGTACCTGCAAATGAGCAAAGTGAGATCTGAGGACACAGCCCTTTATTACTGTGCA
GTCTACTATACTTACGACGTCTGGGCTATGGACTACTGGGGTCAAGGAACCTCAGTCACCGTCTCCTC
AGCTACAACAACAGCCCCATCTGTCTATCCCTTGGTCCCTGGCTGCAGTGACACATCTGGATCCTCGG
TGACACTGGGATGCCTTGTCAAAGGCTACTTCCCTGAGCCGGTAACTGTAAAATGGAACTATGGAGCC
CTGTCCAGCGGTGTGCGCACAGTCTCATCTGTCCTGCAGTCTGGGTTCTATTCCCTCAGCAGCTTGGT
GACTGTACCCTCCAGCACCTGGCCCAGCCAGACTGTCATCTGCAACGTAGCCCACCCAGCCAGCAAGA
CTGAGTNATCAAGAG 3′
Sequence of the light chain of the H44/70 monoclonal antibody
SEQ ID NO:285
5′TGACCCAGTCTCCACTCACTTTTGTCGGTTACCATTGGaCAACCAGCcTCCATcTCTTGCAAGTCA
AGTCAGAGCCTCTTAGATAATGATGGAAAGACATATTTGAATTGGTTGTTACAGAGGCCAGGCCAGTc
TCCAAAGCGCCTAATCTATCTGGTGTCTAAACTGGACTCTGGAGTCCCTGACAGGTTCACTGGCAGTG
GATCAGGGACAGATTTCACACTGAAAATCAGCAGAGTGGAGQCTGAGGATTTGGGAGTTTATTATTGC
TGGCAAGGTACACATTTTCCTCTCACGTTCGGTGCTGGGACCAAGCTGGAGCTGAAACGGGCTGATGC
TGCACCAACTGTATCCATCTTCCCACCATCCAGTGAGCAGTTAACATCTGGAGGTGCCTCAGTCGTGT
GCTTCTTGAACAACTTCTACCCCAAAGACATCAATGTCAAGTGGAAGATTGATGGCAGTGAACGACAA
AATGGCGTCCTGAACAGTTGGACTGATCAGGACAGCAAAGACAGCACCTACAGCATGAGCAGCACCCT
CACGTTGACCAAGGACGAGTATGAACGACATAACAGCTATACCTGTGAGGCCACTCACAAGACATCAA
CTTCACCCATTGTCAAGAGCGG 3′
Sequence of the heavy chain of the H44/70 monoclonal
antibody
SEQ ID NO:286
5′ACAGGTAAGCTGGGGCTTCAGTGAGGATATCCTGTAAGGcTTCtGGcTACACCTTTCACAAGCTAC
TATATACACTGGGTGAAGCAGAGGCCTGGACAGGGACTTGAGTGGATTGGATGGATTTATCCTGGAAA
TGTTAATACTAAGTACAATGAGAAGTTCAAGGGCAAGGCCACACTGACTGCAGACAAATCCTCCAGCA
CAGCCTACATGCAGCTCAGCAGCCTGACCTCTGAGGACTCTGCGGTCTATTTCTGTGCAAGAGGGGGG
GTACGGTATGCTATGGACTACTGGGGTCAAGGAACCTCAGTCACCGTCTCCTCAGAGAGTCAGTCCTT
CCCAAATGTCTTCCCCCTCGTCTCCTGCGAGAGCCCCCTGTCTGATAAGAATCTGGTGGCCATGGGCT
GCCTGGCCCGGGACTTCCTGCCCAGCACCATTTCCTTCACCTGGAACTACCAGAACAACACTGAAGTC
ATCCAGGGTATCAGAACCTTCCCAACACTGAGGACAGGGGGCAAGTACCTAGCCACCTCGCAGGTGTT
GCTGTCTCCCAAGAGCATCCTTGAAGGTTCAGATGAATACCTGGTATGCAAAATCCACTACGGAGGCA
AAAACAGAG 3′
Sequence of the light chain of the H44/78 monoclonal antibody
SEQ ID NO:287
5′CTGACCCAGTTCCACTCACTTtGTcGGtTACCATTGGaCAACCAGCcTCCATcTCTTGCAAGTCAA
GTCAGAGCcTCTTAGATAGTGATGGAAAGACATATTTGAATTGGTTGTTACAGAGGCCAGGCCAGTcT
CCAAAGCGCcTAATCTATCTGGTGTCTAPACTGGACTCTGGAGTCCCTGACAGGTTCACTGGCAGTGG
ATCAGGGACAGATTTCACACTGAAAATCAGCAGAGTGGAGGCTGAGGATTTGGGAGTTTATTATTGCT
GGCAAGGTACACATTTTCCGCTCACGTTCGGTGCTGGGACCAAGCTGGAGCTGAAACGGGCTGATGCT
GCACCAACTGTATCCATCTTCCCACCATCCAGTGAGCAGTTAACATCTGGAGGTGCCTCAGTCGTGTG
CTTCTTGAACAACTTCTACCCCAAAGACATCAATGTCAAGTGGAAGATTGATGGCAGTGAACGACAAA
ATGGCGTCCTGAACAGTTGGACTGATCAGGACAGCAAAGACAGCACCTACAGCATGAGCAGCACCCTC
ACGTTGACCAAGOACGAGTATGAACGACATAACAGCTATACCTGTGAGGCCACTCACAAGACATCAAC
TTCACCCATTGTCAAGAGCG 3′
Sequence of the heavy chain of the H44/78 monoclonal
antibody
SEQ ID NO:288
5′GTCTGGACCTAAGTGGTAAAGCCTGGGGCTTCAGTGAGGATATCCTGCAAGGCTTCTGGcTACACc
TTCACAAGCTACTATATACACTGGGTGAAGCAGAGGCCTGGACAGGGACTTGAGTGGATTGGATGGAT
TTATCCTGGAAATGTTAATACTAAGTACAATGAGAAGTTCAAGGGCAAGGCCACACTGACTGCAGACA
AATCCTCCAGCACAGCCTACATGCAGCTCAGCAGCCTGACCTCTGAGGACTCTGCGGTCTATTTCTGT
GCAAGATCTACTACGGCTAGGGGGTACTTTGACTACTGGGGCCAAGGCACCACTCTCACAGTCTCCTC
AGAGAGTCAGTCCTTCCCAAATGTCTTCCCCCTCGTCTCCTGCGAGAGCCCCCTGTCTGATAAGAATC
TGGTGGCCATGGGCTGCCTGGCCCGGGACTTCCTGCCCAGCACCATTTCCTTCACCTGGAACTACCAG
AACAACACTGAAGTCATCCAGGGTATCAGAACCTTCCCAACACTGAGGACAGGGGGCAAGTACCTAGC
CACCTCGCAGGTGTTGCTGTCTCCCAAGAGCATCCTTGAAGGTTCAGATGAATACCTGGTATGCAAAA
TCCACTACGGAGGCAAAAACAGAG 3′

We claim:

1. A mimotope of a surface L3,7,9 LOS of N. meningitidis, said mimotope comprising a peptide epitope obtainable by screening a peptide library with a monoclonal antibody selected from a group consisting of: 4BE12C10; H44/24; H44/58; H44/70; and H44/78.

2. A mimotope of a surface L3,7,9 LOS of N. meningitidis, said mimotope being antigenically cross-reactive with a monoclonal antibody selected from a group consisting of: 4BE12C10; H44/24; H44/58; H44/70; and H44/78.

3. The mimotope according to claim 1 or claim 2, which comprises a peptide epitope contained within any one of the peptides of SEQ ID NO: 1-140, 289-296, or retro sequences thereof.

4. The mimotope according to claim 3, wherein the peptide epitope is present in a nonapeptide.

5. The mimotope according to claims 3-4, which comprises any one of the peptides of SEQ ID NO: 1-140, 289-296, or retro sequences thereof, or modifications of the peptides or retro sequences which retain cross-reactivity with a monoclonal antibody selected from a group consisting of: 4BE12C10; H44/24; H44/58; H44/70; and H44/78.

6. The mimotope of claims 3-5, wherein the peptide epitope comprises the amino acid sequence PP[Y or F or W].

7. The mimotope of claim 6, wherein the peptide epitope comprises the amino acid sequence PP[Y or F or W]D.

8. The mimotope of claim 7, wherein the peptide epitope comprises the amino acid sequence PPYD.

9. The mimotope of claims 3-5, wherein the peptide epitope comprises the amino acid sequence WY.

10. The mimotope of claim 9, wherein the peptide epitope comprises the amino acid sequence WYXXP.

11. The mimotope of claims 3-5, wherein the peptide epitope comprises the amino acid sequence [Y or W]XY.

12. The mimotope of claims 3-11, wherein said mimotope comprises an oligopeptide, comprising said peptide epitope, which is structurally more constrained than an unsubstituted linear form of the oligopeptide.

13. The mimotope according to claim 12, wherein the oligopeptide comprises a cyclic peptide encompassing the peptide epitope.

14. The mimotope according to claim 13, wherein the cyclic peptide comprises a cyclised portion which comprises an amino acid sequence, the terminal amino acids of which are linked together by a covalent bond.

15. The mimotope according to claim 14, wherein the cyclic peptide is any one of the peptides of SEQ ID NO: 141-280, 297-301, or retro sequences thereof.

16. The mimotope according to any one of the preceding claims, wherein a carrier is conjugated to the mimotope to enhance the immunogenicity thereof.

17. The mimotope according to claim 16, wherein the carrier comprises an immunogenic protein.

18. A vaccine comprising a mimotope according to any one of the preceding claims and a suitable excipient or diluent.

19. A vaccine against serogroup B, C, Y, or W-135 meningococci, which comprises a mimotope of a surface L3,7,9 LOS of N. meningitidis and a mimotope of a surface L2 LOS of N. meningitidis.

20. The vaccine according to claim 19, wherein the mimotopes are antigenically cross-reactive with a monoclonal antibody of high specificity to the respective surface LOS.

21. The vaccine according to claim 19 or claim 20, wherein the mimotopes each comprise a peptide epitope.

22. The vaccine according to claim 21, wherein the peptide epitopes are obtainable by screening a peptide library with the respective monoclonal antibodies of claim 20.

23. The vaccine of claim 21 or 22, wherein the L3,7,9 and the L2 mimotopes are contained within the same molecule.

24. The vaccine according to claims 21-23, wherein the mimotope of a surface L3,7,9 LOS is the mimotope of claims 2-17.

25. The vaccine according to claims 21-23, wherein the mimotope of a surface L3,7,9 LOS comprises a peptide selected from:
IHRQGIH; HIGQRHI; LPARTEG; GETRAPL; APARQLP; PLQRAPA; KQAPVHH; HHVPAQK; LQAPVHH; HHVPAQL; LPSIQLP; PLQISPL; NELPHKL; LKHPLEN; KSPSMTL; LTMSPSK; AGPLMLL; LLMLPGA; WSPILLD DLLIPSW; LSMHPQN; NQPHMSL; HSMHPQN NQPHMSH; SMYGSYN; NYSGYMS; TNHSLYH; HYLSHNT; HAIYPRH; HRPYIAH; TTYSRFP; PFRSYTT; TDSLRLL; LLRLSDT; SFATNIP; and PINTAFS.

26. The vaccine according to claim 19-25, wherein the mimotope of a surface L2 LOS is antigenically cross-reactive with F1-5H 5/ID9 monoclonal antibody.

27. A vaccine against serogroup A meningococci, which comprises a mimotope of a surface L3,7,9 LOS of N. meningitidis and a mimotope of a surface L10 LOS of N. meningitidis.

28. The vaccine according to claim 27, wherein the mimotope of a surface L10 LOS is antigenically cross-reactive with a monoclonal antibody of high specificity to the L10 LOS.

29. The vaccine according to claim 28, wherein the monoclonal antibody is 5B4-F9-B10.

30. The vaccine of claim 28 or 29, wherein the mimotope of a surface L10 LOS comprises a peptide epitope.

31. A meningococcal vaccine comprising a mimotope of a surface L3,7,9 LOS of N. meningitidis, a mimotope of a surface L10 LOS of N. meningitidis, and a mimotope of a surface L2 LOS of N. meningitidis.

32. A meningitis vaccine comprising the vaccine of claim 31, and a conjugated H. influenzae b capsular polysaccharide.

33. A H44/24 hybridoma deposited under the Budapest Treaty patent deposit at ECACC on 22/9/00 with Provisional Accession Number 92209.

34. A H44/58 hybridoma deposited under the Budapest Treaty patent deposit at ECACC on 22/9/00 with Provisional Accession Number 92210.

35. A H44/70 hybridoma deposited under the Budapest Treaty patent deposit at ECACC on 22/9/00 with Provisional Accession Number 92211.

36. A H44/78 hybridoma deposited under the Budapest Treaty patent deposit at ECACC on 22/9/00 with Provisional Accession Number 92212.

37. A monoclonal antibody obtainable from any of the hybridomas of claims 33-36.

38. Use of the monoclonal antibodies of claim 37 in the identification of mimotopes of N. meningitidis L3,7,9 LOS.

39. A pharmaceutical composition comprising the monoclonal antibody of claim 37.

40. A mimotope as described in claims 1-17, or a vaccine as described in claims 18-32, or a monoclonal antibody as described in claim 37 for use as a medicament.

41. Use of the mimotope of claims 1-17, or the vaccine of claims 18-32, or the monoclonal antibody of claim 37 in the manufacture of a medicament for the treatment or prevention of meningococcal disease.

42. A method of manufacturing a vaccine comprising the manufacture of a mimotope as claimed in any one of claims 1 to 17, and formulating the mimotope with an adjuvant.

43. A method for treating a patient suffering from or susceptible to meningococcal disease, comprising the administration of the mimotope of claims 1-17, or the vaccine of claims 18-32, or the monoclonal antibody of claim 37, to the patient.

44. A DNA sequence encoding the mimotope of claims 1-16.

45. A diagnostic assay for meningococcal infection comprising the use of the mimotope of claims 1-16 to detect antibodies against L3,7,9 LOS in the serum of a patient.

46. A diagnostic assay for meningococcal infection comprising the use of the monoclonal antibody of claim 37 to detect the presence of L3,7,9 immunotype meningococcus in a sample from a patient.

47. A DNA sequence encoding a CDR region encoded by the DNA sequences of SEQ ID NO:281-288.
